(19) United States
(12) Patent Application Publication
(10) Pub. No.: US 2021/0156846 A1
(43) Pub. Date: May 27, 2021

(54) MICROFLUIDIC PLATFORM FOR TARGET AND BIOMARKER DISCOVERY FOR NON-ALCOHOLIC FATTY LIVER DISEASE

(71) Applicant: Javelin Biotech, Inc., Woburn, MA (US)

(72) Inventors: Murat Cirit, Cambridge, MA (US); Begum Alaybeyoglu, Woburn, MA (US); Nathan Brent Sorensen, Woburn, MA (US); John Wayne Rumsey, Woburn, MA (US)

(21) Appl. No.: 17/104,708

(22) Filed: Nov. 25, 2020

Related U.S. Application Data
(60) Provisional application No. 62/940,551, filed on Nov. 26, 2019.
(60) Provisional application No. 63/074,717, filed on Sep. 4, 2020.

(51) Int. Cl. G01N 33/50; C12Q 1/6883; G16B 5/00

(52) U.S. Cl. CPC G01N 33/5067; C12Q 1/6883; C12Q 2600/158; C12Q 2600/106; G16B 5/00

(57) ABSTRACT

A method for developing stratified medicine for nonalcoholic fatty liver disease (NAFLD includes obtaining a microphysiological system (MPS) comprising a liver tissue cytoarchitecture, adipose tissue, or both. The method includes inducing metabolic dysfunction representing NAFLD in the liver or adipose tissue of the MPS. The method includes generating, based on inducing the metabolic dysfunction, transcriptomics data for the MPS. The method includes applying a drug to the MPS using a dosing regimen. The method includes monitoring changes in the transcriptomics data based on applying the drug. The method includes generating a model relating the changes in the transcriptomics data to the dosing regimen of the drug.

### CLAIM OF PRIORITY

This application claims priority under 35 U.S.C. § 119(e) to provisional U.S. Patent Application Ser. No. 62/940,551, filed on Nov. 26, 2019, and Application Ser. No. 63/074,717, filed on Sep. 4, 2020, the entire contents of each of which are hereby incorporated by reference.

### FIELD OF THE INVENTION

This disclosure generally relates to microfluidic devices. More specifically, this disclosure relates to target and metabolomics-based biomarker discovery platforms for diagnosis of non-alcoholic fatty liver disease (NAFLD).

### BACKGROUND

Non-alcoholic fatty liver disease (NAFLD) is a progressive disease from a steatotic liver to steatohepatitis in non-alcohol or low-alcohol consuming individuals. NAFLD begins as nonalcoholic fatty liver (NAFL) and progresses to nonalcoholic steatohepatitis (NASH), cirrhosis and hepatocellular carcinoma (HCC) over time. NAFLD is estimated to affect 30% of the global population and is underdiagnosed in the majority. Furthermore, NAFLD is comorbid with obesity and diabetes, and rates are expected to rise proportional to the obesity epidemic. In the majority of patients, NAFLD develops asymptomatically and is only discovered in the late stages of the disease after NAFL has progressed to NASH and liver fibrosis, cirrhosis or HCC has taken hold. Efforts to ameliorate NAFLD through pharmacological intervention have been unsuccessful. These scenarios highlight the need for early detection of NAFLD, both in health practices and as a companion diagnostic in clinical trials.

Current diagnostic approaches for NAFLD include histological examination of a liver biopsy. This is a highly invasive procedure. This procedure is rarely considered before serious symptoms arise and is difficult to justify routine monitoring of disease progression. Determined by histological features, a NAFLD activity score (NAS) system is used, in part, to score the severity of hepatitis and steatosis grade. Alone, NAS does not correlate with disease progression, does not predict outcomes, nor is accurate enough to distinguish between NAFL and NASH.

It is desirable to develop a noninvasive method (e.g. sampling blood or urine, etc.) for monitoring the progression of NAFLD, or preventative screens for high-risk potential NAFLD patients. Efforts in noninvasive biomarker discovery for quantitative diagnosis of NAFL and NASH and plasma components have been associated with disease severity as potential predictors of disease progression. Common serum biomarkers of liver diseases include alanine aminotransferase (ALT), aspartate aminotransferase (AST), albumin, uric acid, bilirubin, triglycerides, cholesterol (HDL & LDL), glucose, but no single factor provides a prediction or diagnosis for NAFLD. Some combinations of serum biomarkers have been loosely correlated with NAFLD/NASH progression, including free fatty acid species, and AST and ALT ratios. Utilization of -omics contributes to the knowledgebase of NAFLD pathogenesis through revealed molecular signatures, which can offer diagnostic markers only after excessive validation and replication. In fact, a panel of lipids measured in the serum of NAFLD patients was found to be predictive of their NAS values, correctly classifying patients according to NAFLD stages, while a panel of plasma metabolites was reported to differentiate between NASH and steatosis patients.

Noninvasive biomarker discovery performed in the clinic has significant challenges. NAFLD progresses non-linearly and can take decades to develop into a life-threatening condition. Frequently testing large cohorts of patients on this time scale is inherently difficult. Routine liver biopsies are cost prohibitive, and ethically questionable on asymptomatic patients. Over time, the effect of disease-relevant lifestyle choices (such as diet and exercise) or co-morbidities on NAFLD progression could significantly confound a long-term study. Furthermore, it is difficult to directly correlate quantitative biomarker measures to subjective histological and patient response scales, especially for this slow moving chronic disease. Without innovative non-clinical approaches, it is likely that discovery, validation and implementation of routine biomarker testing would be high risk and take a long period of time (e.g., decades).

Additionally, disease-modifying biomarkers (called targets) include a subset of differentially expressed biomarkers that can modify a disease. For example, the targets may cure the disease, slow the disease progression, or have some other such effect. The identified disease-modifying biomarkers can be evaluated for their disease modifying potential with small molecules or CRISPR analysis. If these disease-modifying biomarkers modify the disease, they are called disease-modifying targets.

### SUMMARY

This disclosure describes a preclinical target discovery and drug development MPS platform for stratified medicine in NAFLD.

The platform can enable one or more technical advantages. The platform is configured to reduce preclinical discovery timelines for NAFLD. Currently, a lack of effective biomarkers and the multitude of trial failures in the NAFLD space show that current preclinical and translational efforts are not adequate. To overcome these significant challenges in the clinic, a preclinical microfluidic platform, also called a microphysiological system (MPS), is developed to study druggable targets and drug efficacy on NAFLD is becoming possible. The preclinical MPS accommodates high-throughput experimentation and also recapitulates tissue function and organization. Using primary cells, MPSs perform significantly better in recreating an in vivo environment and in predicting in vivo outcomes than traditional cell culture systems (i.e. immortalized cell lines on plastic cultureware). Generally, MPSs are tailored to fit specific purposes, incorporating multiple cell types, cellular matrices, microfluidics and/or multiple tissue compartments.

Another advantage for MPSs described herein is that the MPS are configured for integration with computational systems biology. In vitro to in vivo translation is used for translation of pharmacodynamics models. In addition, combining -omics technologies with MPSs improves translational relevance. The large -omics data sets provide greater molecular insight relative to standard in vitro assays. The MPSs described herein utilize metabolomics and transcriptomics to classify/stage NAFLD (NAFL & NASH) in vitro and translate our findings to the clinical applications. MPS and -omics technologies are integrated to accelerate metabolite-based biomarker discovery for a noninvasive NAFLD diagnostic using systems biology analysis and a multi-donor multicellular liver MPS.

The preclinical target discovery and drug development MPS platforms described herein provide an approximation of the complex human physiology and disease spectrum reproduction of the clinical disease spectrum (steatosis, steatohepatitis, ballooning, and fibrosis) and metabolic features (IR and obesity) of human NAFLD. Animal in vivo models (e.g. dietary, genetic, and chemical) and human in vitro models have been insufficient to reproduce these aspects of NAFLD. For example, the MPSs are able to recapitulate the cytoarchitectural and chemical signaling complexity of multi-organ nature of the NAFLD progression. Additionally, MPS is relatively low-cost in comparison with animal in vivo studies, can provide high throughput analysis not possible with animal in vivo studies, and provides more relevant results than animal in vivo studies. Specifically, the MPSs described herein enable reconstruction of multi-cellular cytoarchitectures of human tissues and pathophysiologies in a high-content or high-throughput manner. Additionally, subpopulations are established with the MPSs described herein to discover drugs for various subpopulation (e.g., a stratified medicine approach), potentially overcoming the bottleneck of evaluating population responses at preclinical stage (e.g., prior to a phase II or a phase III clinical trial stage).

The preclinical target discovery and drug development MPS platform for stratified medicine in NAFLD can provide one or more additional advantages. The MPS integrates several workflows to identify disease modifying biomarkers and/or targets and combination therapies in various sub-populations. The MPS recapitulates NAFL-NASH phenotypes and disease progression using tissue engineered, human-based MPSs on the liver-adipose axis. The emerging multi-scale data (phenotypic and multi-omics) are processed with a NAFLD-Net computational model to identify molecular dysfunctions for various disease phenotypes (steatosis, NASH without fibrosis and NASH with fibrosis) and to rank order the potential therapeutic targets associated with disease phenotypes. The disease-modifying potential of the rank-ordered targets is experimentally evaluated using functional genomics approaches enabled by the MPSs, such as CRISPR gene editing. Additionally, the MPSs described herein enable analysis of various NAFLD high-risk populations based on the known NAFLD epidemiology, such as metabolic syndrome, PNPLA3 mutation, and pre-menopausal and post-menopausal sex-hormone changes. The MPSs enable mechanisms of action-based drug discovery but also stratified medicine at the preclinical discovery stage.

The one or more advantages described can be enabled by one or more aspects or embodiments of the platform.

In an embodiment, a process for developing stratified medicine for nonalcoholic fatty liver disease (NAFLD) includes obtaining a first microphysiological system (VIPS) comprising a liver tissue cytoarchitecture. The process includes obtaining a second MPS comprising an adipose tissue cytoarchitecture, the first MPS and the second MPS each comprising a compartment in a common fluidic system, wherein crosstalk occurs between the first MPS and the second MPS based on fluid flow in the common fluidic system. The process includes inducing metabolic dysfunction representing NAFLD in each of the first MPS and the second MPS. The process includes generating, based on inducing the metabolic dysfunction, transcriptomics data for each of the first MPS and the second MPS. The process includes applying a drug to the first MPS and the second MPS using a dosing regimen. The process includes monitoring changes in the transcriptomics data based on applying the drug. The process includes generating a model relating the changes in the transcriptomics data to the dosing regimen of the drug.

In some implementations, the drug comprises one or more of small and large molecules configured to modulate activity of disease-relevant signaling pathways. In some implementations, the one or more of small and large molecules comprise one or more of rosiglitazone (PPARγ), elafibranor (PPARα and PPARβ/δ), obeticholic acid (OCA) (FXR), and cenicriviroc (CCR2-5).

In some implementations, the drug comprises CRISPR short guide RNAs (sgRNAs) that modulate an activity or expression of disease-relevant signaling pathways.

In some implementations, the sgRNAs comprise one or more of FXR, PPARα, PPARβ/δ, PPARγ, and CCR2-5. In some implementations, the dosing regimen comprises applying the drug at five concentrations spanning a nano-molar to milli-molar range.

In some implementations, inducing metabolic dysfunction representing NAFLD in each of the first MPS and the second MPS comprises one or more of: inducing one or more of insulin resistance (IR), excessive de novo gluconeogenesis and lipogenesis, and dysregulated hepatokine signaling in the first MPS; and inducing one or more of IR, increased lipolysis, and dysregulated adipokine signaling in the second MPS. In some implementations, inducing the metabolic dysfunction comprises: characterizing one or more of phenotypic, biomarker, and transcriptomic signatures of NAFLD pathology; and determining, a physiological relevance of one or more phenotypes, biomarkers, or transcriptomics of NAFLD.

In some implementations, the process includes applying the model to one or more stratified patient subpopulations based on differential biological mechanisms for each stratified patient subpopulation. In some implementations, the differential biological mechanism comprises one of a high-risk genetic single nucleotide polymorphism or a gender-specific hormone. In some implementations, the process includes generating, based on applying the model, one or more of phenotypic, transcriptomic, and metabolomic datasets establishing a molecular characterization of each stratified patient subpopulation.

In some implementations, the process includes connecting the first MPS and the second MPS by milli-fluidic recirculation to facilitate one or more of a hepatokine, an adipokine, and a cytokine crosstalk between the first MPS and the second MPS; and scaling each of the first MPS and the second MPS are each scaled based on a human physiology for one or more of the hepatokine, the adipokine, and the cytokine crosstalk. In some implementations, the human physiology represents comprises an oxygen-dependent liver metabolic zonation profile.

In an embodiment, a method for developing stratified medicine for nonalcoholic fatty liver disease (NAFLD) includes obtaining a first microphysiological system (MPS) comprising a liver tissue cytoarchitecture. The process includes obtaining a second MPS comprising an adipose tissue cytoarchitecture. The process includes combining the first MPS and the second MPS into one recirculating platform including two compartments connected by connecting outlets of each of the first and second MPS into a mixing compartment. The process includes seeding pre-adipocytes into the second MPS, wherein the pre-adipocytes are differentiated into adipocytes. The process includes seeding hepatocytes and stellate cells into the first MPS. The process includes applying liver sinusoidal endothelial cells (LSECs) and Kupffer cells into the first MPS. The process includes switching the first MPS and the second MPS to either a medium including disease-inducing factors or to a physiologically healthy medium. The process includes monitoring each of the first MPS and the second MPS for a disease progression. The process includes extracting RNA or intracellular metabolites from the first MPS or the second MPS. The process includes determining, based on extracting, one or more of a transcriptomic and metabolomic profile associated with the disease progression.

In some implementations, the disease progression comprises nonalcoholic fatty liver (NAFL), nonalcoholic steatohepatitis (NASH)-fibrosis, or NASH and fibrosis. In some implementations, the process includes reconstructing one or more tissue-specific models for liver and adipose from global human metabolic network models. In some implementations, the process includes integrating one or more validated and functional liver-specific and adipose-specific genome-scale metabolic models (GEMs) through a media compartment connected to each of the first MPS and the second MPS; generating a liver-adipose GEM representing a healthy metabolic state; and comparing the healthy metabolic state to a diseased state. In some implementations, the diseased state comprises one or more of steatosis, steatohepatitis, and NASH with fibrosis phenotypes.

In some implementations, the process includes identifying a target from the GEM; perturbing the target with small molecules; and evaluating a change in a phenotype of the target to determine whether NAFLD is improved.

In some implementations, a method for developing stratified medicine for nonalcoholic fatty liver disease (NAFLD) includes obtaining a microphysiological system (MPS) comprising a liver tissue cytoarchitecture. The process includes inducing metabolic dysfunction representing NAFLD in the liver tissue of the MPS. The process includes generating, based on inducing the metabolic dysfunction, transcriptomics data for the MPS. The process includes applying a drug to the MPS using a dosing regimen. The process includes monitoring changes in the transcriptomics data based on applying the drug. The process includes generating a model relating the changes in the transcriptomics data to the dosing regimen of the drug.

In an embodiment, a system includes a first microphysiological system (MPS) comprising a liver tissue cytoarchitecture; a second MPS comprising an adipose tissue cytoarchitecture; the first MPS and the second MPS each comprising a compartment in a common fluidic system, wherein crosstalk occurs between the first MPS and the second MPS based on fluid flow in the common fluidic system; wherein the system is configured for induced metabolic dysfunction representing NAFLD in each of the first MPS and the second MPS, for generating, based on inducing the metabolic dysfunction, transcriptomics data for each of the first MPS and the second MPS, for applying a drug to the first MPS and the second MPS using a dosing regimen, for monitoring changes in the transcriptomics data based on applying the drug, and for generating a model relating the changes in the transcriptomics data to the dosing regimen of the drug

The details of one or more embodiments of these systems and methods are set forth in the accompanying drawings and the description to be presented. Other features, objects, and advantages of these systems and methods are apparent from the description and drawings, and from the claims.

### BRIEF DESCRIPTION OF THE DRAWINGS

FIGS. 1A-1E are block diagrams and graphs illustrating example MPS platforms and associated data for preclinical drug discovery system for developing treatments (e.g., drugs) for NAFLD.

FIGS. 2A-3C show example data representing a result of a process for establishing metabolically dysfunctional MPSs of FIGS. 1A-1E.

FIG. 4 shows example results of CRISPR-mediated activation and knock-out of genes in the MPS for MPSs of FIGS. 1A-1E.

FIG. 5 shows liver-adipose crosstalk in a multi-compartment MPS.

FIG. 6A shows an example workflow for a preclinical target discovery.

FIG. 6B shows a multi-omics analysis and pathway-based integration.

FIG. 7 shows examples of cytokine panel data.

FIGS. 8A-8C show images representing green fluorescent protein signal intensity in untransfected liver spheroids.

FIGS. 9A-9B are flow diagrams illustrating example processes for analysis of MPS data for preclinical drug discovery system for developing treatments (e.g., drugs) for NAFLD.

FIG. 10 is a block diagram of an example computer system used to provide computational functionalities associated with described algorithms, methods, functions, processes, flows, and procedures described in the present disclosure.

FIG. 11 is a diagram illustrating an example computer system configured to execute a machine learning model.

### DETAILED DESCRIPTION

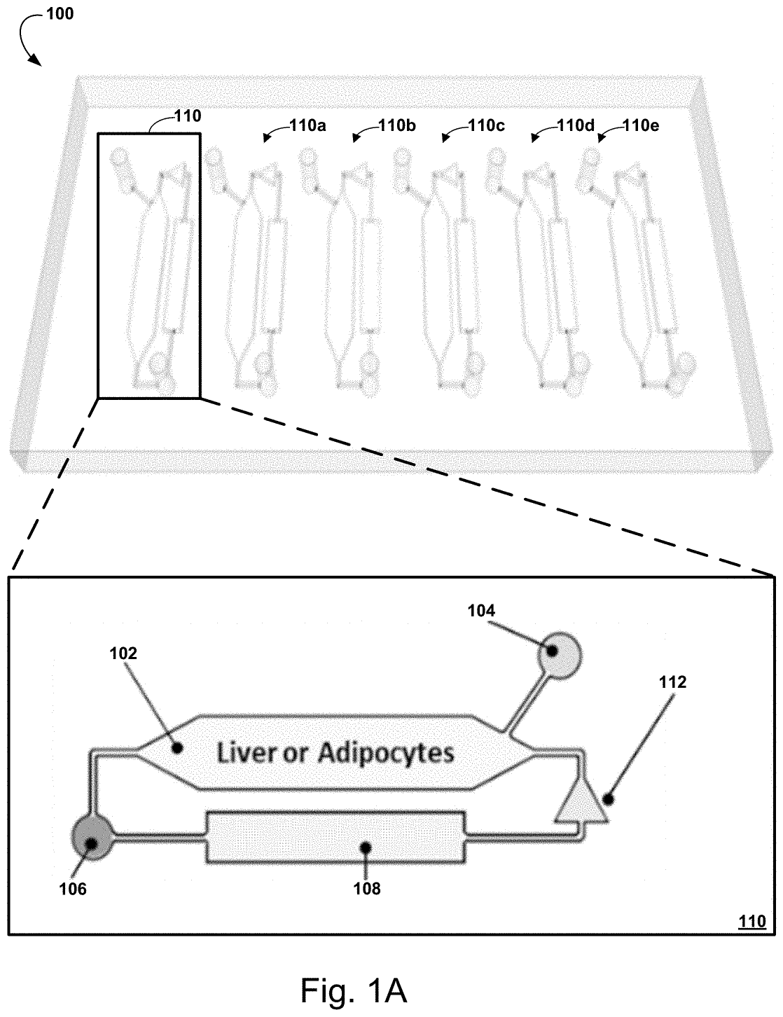

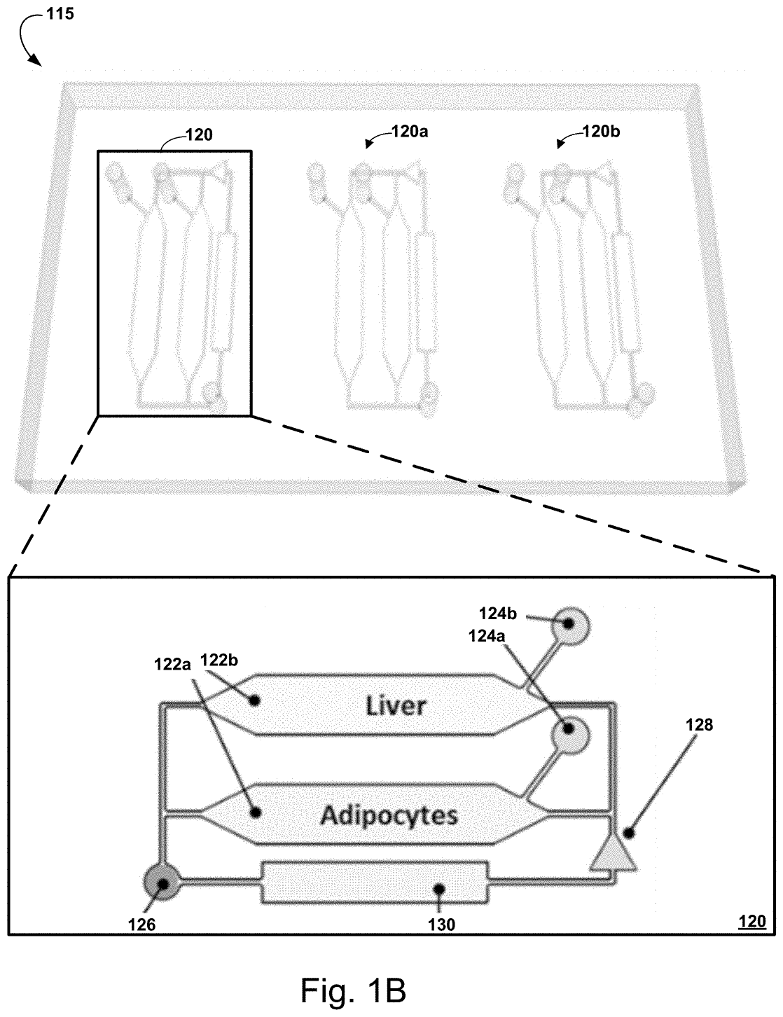

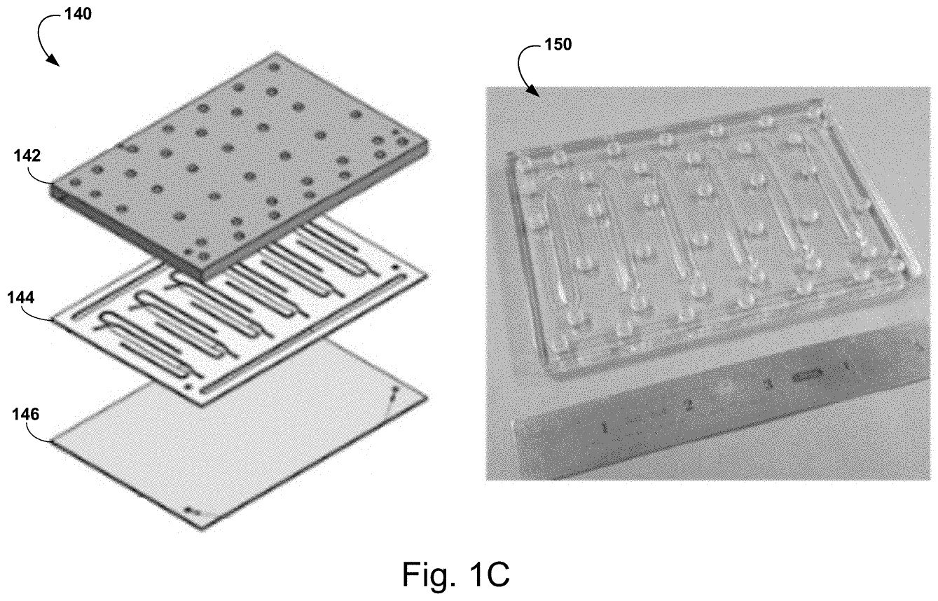

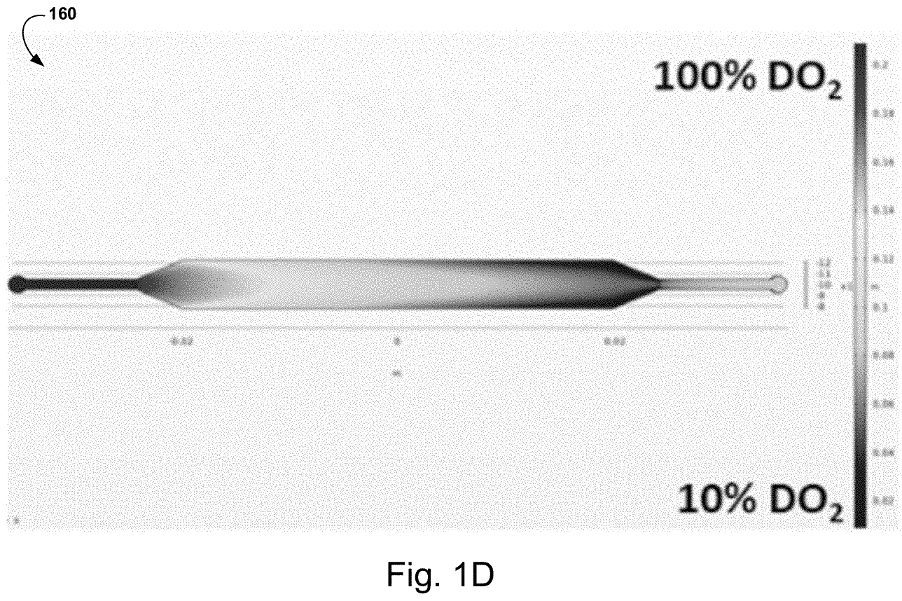

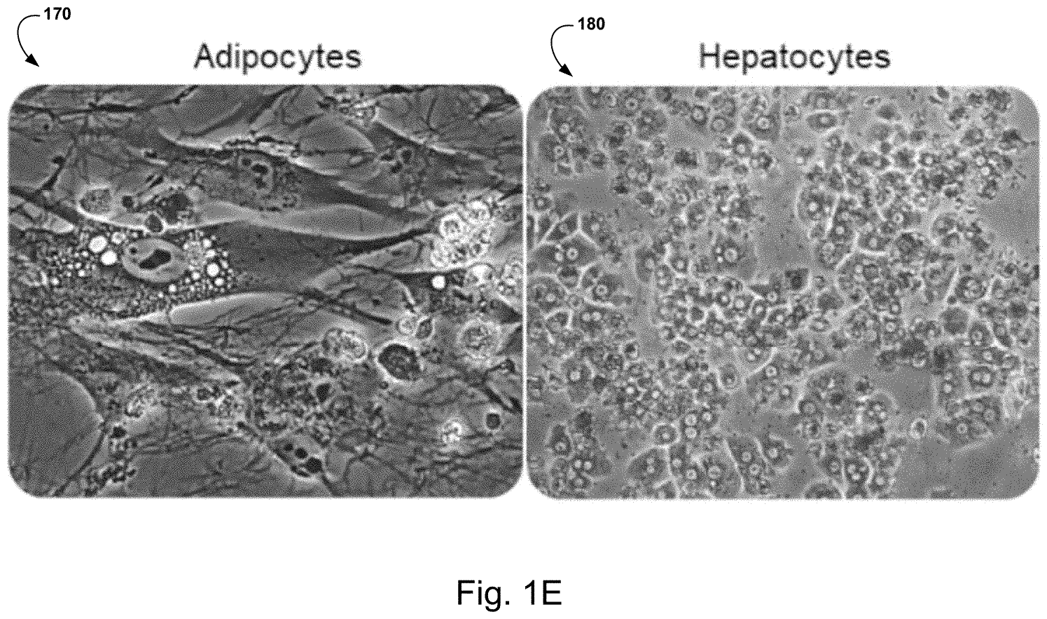

This disclosure describes preclinical target discovery and drug development MPS platform for stratified medicine in NAFLD. FIG. 1A is a block diagram illustrating an example hardware platform 100 for hosting one or more MPSs (e.g., MPS 110) configured to emulate liver or adipose organ functionality. MPS 110 is configured to enable preclinical drug discovery and assist in developing treatments for NAFLD. MPS 110 enables long-term tissue mono- & co-culture (e.g., longer than 4 weeks). MPS 110 generally includes a cell chamber 102 including liver or adipose tissue. MPS 110 includes a cell seeding port 104 for introducing the liver cells or adipose cells to the cell chamber 102. MPS 110 includes a media access port 106 for accessing fluid media in the MPS, either for fluid media removal or introduction to the MPS, as subsequently described. A fluid mixing and re-oxygenation chamber 108 is configured to mix the fluid media in the MPS 110 and add oxygen to the fluid media. A mircopump 112 is included in a fluid circuit to circulate media through the cell chamber 102 and the mixing and re-oxygenation chamber chamber 108. This fluid circuit can be configured to introduce substances (e.g., drugs or other substances) to the cell chamber 102 to emulate functionality of in vivo human tests, as subsequently described.

The hardware platform can include multiple additional MPSs 110a-e. MPSs 110a-e can be additional instances of MPS 110. The MPSs 110 and 110a-e operate independently from one another (e.g., for high-throughput screening) for testing compounds, such as drugs, in simulated human in vivo-relevant mircoenvironments. In some implementations, one or more of the MPSs 110a-e can be combined to link multiple different tissues or cell cultures together.

Similarly, FIG. 1B is a block diagram illustrating an example hardware platform 115 for hosting one or more MPSs (e.g., MPS 120) configured to emulate liver and adipose cells in separate cell chambers. MPS 120 is configured to enable preclinical drug discovery and assist in developing treatments for NAFLD. MPS 120 enables long-term tissue mono- & co-culture (e.g., longer than 4 weeks). MPS 120 generally includes two cell chambers 122a and 122b. Chamber 122a includes adipose tissue. Chamber 122b includes liver tissue. Chambers 122a and 122b are configured to be in parallel with one another in the fluid loop of MPS 120. In this example, the system 120 can be called a multi-MPS system because there are multiple cell compartments 122a-b. For simplicity, each instance of system 120 is referred to as an MPS 120 in this document, even though the system 120 a multi-compartment (and therefore multi-MPS) system.

MPS 120 includes two cell seeding ports 124a and 124b. The cell seeding ports 124a-b are for introducing the adipose tissue or liver cells to the cell chambers 122a and 122b, respectively. MPS 120 includes a media access port 126 for accessing fluid media in the MPS, either for fluid media removal or introduction to the MPS, as subsequently described. A fluid mixing and re-oxygenation chamber 130 is configured to mix the fluid media in the MPS 120 and add oxygen to the fluid media. A mircopump 128 is included in a fluid circuit to circulate media through the cell chambers 122a-b and the mixing chamber 130. This fluid circuit can be configured to introduce substances (e.g., drugs or other substances) to the cell chambers 122a-b to emulate functionality of in vivo human tests, as subsequently described. The hardware platform can include multiple additional MPSs 120a-b. MPSs 120a-b can be additional instances of MPS 120. The MPSs 120 and 120a-b operate independently from one another (e.g., for high-throughput screening) for testing compounds, such a drugs, in simulated liver in vivo mircoenvironments. In some implementations, one or more of the MPSs 120a-b can be combined to link multiple different tissues or cell cultures together.

The MPS 120 is an integrated liver-adipose crosstalk system. The MPS 120 includes both the liver compartment 122b and the adipose tissue compartment 122a connected by a milli-fluidic recirculation to facilitate hepatokine, adipokine, and cytokine crosstalk. The compartment 122a-b geometries are estimated using a multi-functional scaling methodology. The geometries are optimized to establish an oxygen-dependent liver metabolic zonation profile which is relevant to NAFL-NASH disease progression. For example, a relationship between severity and zonal location of steatosis to the presence of NASH include that there is an increased disease severity (e.g., fibrosis and ballooning) in zone 3 (perivenous—low oxygen) compared to zone 1 (periportal—high oxygen). Briefly, for the multi-functional scaling of liver-adipose MPS 120, a computational model for the adipose-liver axis is focused on crosstalk molecules, such as hepatokines (liver-function), adipokines (adipose-function) and cytokines (both adipose and liver). The measured -kine secretion rates, subsequently described, are the inputs for these models, while additional literature data are used as either as input parameters (e.g. -kine receptor and Kd values) or the output functions (such as -kine plasma and/or portal vein concentrations, in vitro -kine dose response curves). A multi-functional scaling algorithm optimizes the relative tissue sizes (e.g., the input function) to fit a desired the -kine concentrations (e.g., the output function) at which a biological effect is observed. For example, leptin concentrations (e.g., secreted from the adipose MPS) are at physiologically relevant concentrations to activate leptin receptors in the liver MPS. This approach enables scaling relative MPS sizes to create a physiologically relevant crosstalk microenvironment in MPS 120. The algorithmic solutions are approximations for relative tissue sizes.

The MPSs 110, 120 are configured for disease modeling by enabling emulation of tissues in controlled environments. Here, MPSs 110, 120 refers to either MPS 110, MPS 120, or both MPS 110 and MPS 120, either individually or in any combination. For performing a test, there can be an induction of disease in the MPSs 110, 120, hosted by the prepared liver and/or adipose tissues. The disease progression can be monitored over long periods of time. In some implementations, the MPSs 110, 120 can include cells from diseased patients. Many different mechanisms can be used for on-chip disease induction, as subsequently described.

The MPSs 110, 120 are configured for disease characterization. For example, multi-scale assays can be performed using the MPSs 110, 120. The MPSs 110, 120 enable evaluation of cell construct and tissue construct functions. The MPSs 110, 120 enable the system to compare healthy phenotypes and disease phenotypes. The MPSs 110, 120 enable acquisition of data from pre-determined phenotypic metrics and -omics analysis, as subsequently described.

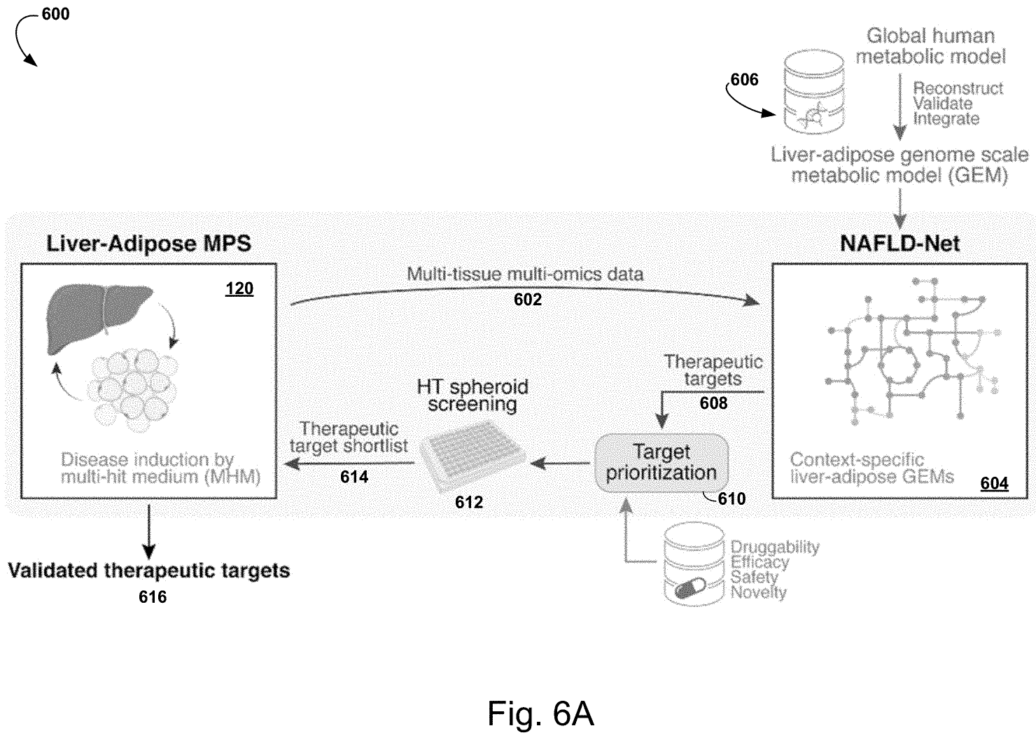

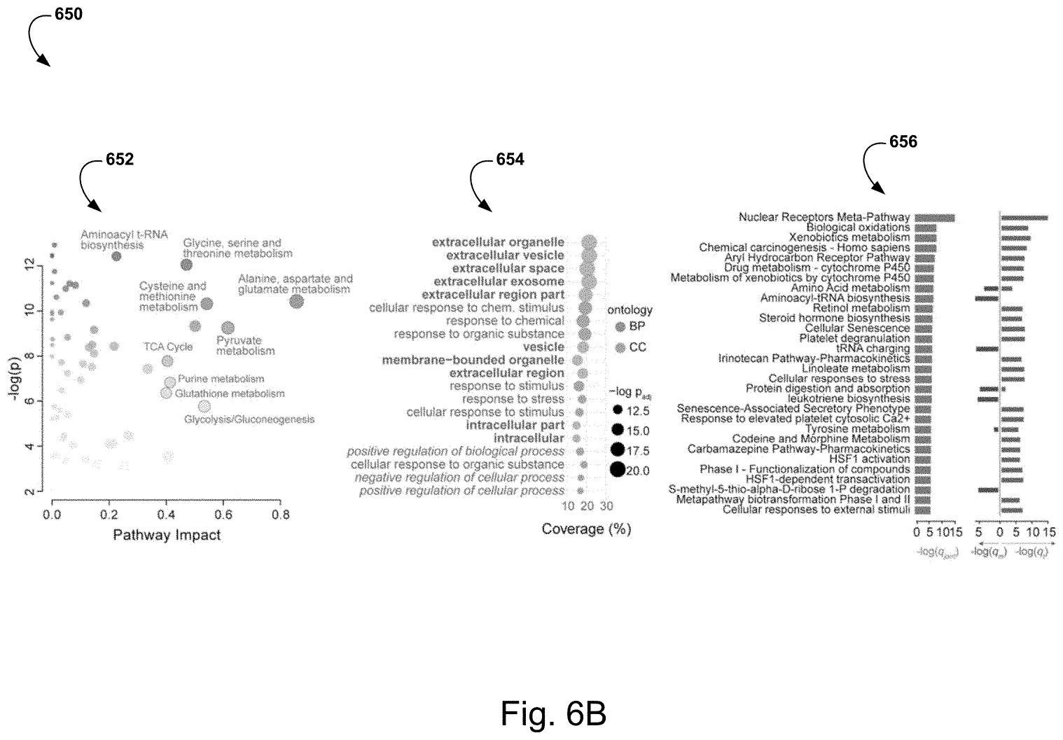

A computing system (not shown) uses data developed from the MPSs 110, 120 to combine MPS models with model-informed drug discovery (MIDD) methodologies in a processing workflow. The processing workflow, subsequently described in relation to FIG. 6A can be used to develop an understanding of NAFLD diagnostic and response biomarkers. The computational modeling is performed for target (e.g., drugs or drug combinations) discovery using the MPS data of the MPSs 110, 120 and SB and quantitative systems pharmacology (QSP) based models. These models are configured to identify molecular abnormalities for diseased cells or tissues. The models link the molecular data to the phenotypic data. Here, phenotypic data can include clinical information regarding disease symptoms, as well as relevant demographic data (if applicable), such as age, ethnicity and sex.

The MPSs 110, 120 are each configured to emulate portions of the liver or fatty tissue of a human. Data obtained from this emulation is used by the computing system model to validate a physiological relevance and identify molecular changes of various NAFLD phenotypes. For example, the MPS 110, 120 can be configured to identify and validate cell-specific targets for NAFLD. The MPSs 110, 120 can be referred to as organ constructs and organ-on-chips (OOC). In some implementations, the cell chambers of the MPSs 110, 120 are called compartments.

The MPSs 110, 120 are configured to recapitulate metabolic dysfunction and NAFLD. Metabolic syndrome (MetS) and/or NAFLD can result in organ dysfunction in the liver, including insulin resistance (IR), excessive de novo gluconeogenesis and lipogenesis, and dysregulated hepatokine signaling. MetS and NALFD can result in visceral adipose tissue (VAT), including IR, increased lipolysis, and dysregulated adipokine signaling. The MPSs 110, 120 can include a liver MPS including primary human hepatocytes, Kupffer cells, stellate cells, liver sinusoidal endothelial cells. The MPSs 110, 120 can include an adipose MPS including visceral adipocytes. Using a defined medium (MHM, multi-hit medium) that mimics the multi-hit pathology (hyperinsulinemia, hyperglycemia, hyperlipidemia, and proinflammation) of NAFLD and formulated using clinically-relevant concentrations, the MPSs 110, 120 are analyzed to characterize phenotypic, biomarker, and transcriptomic signatures of disease pathology and establish the physiological relevance of the disease phenotypes compared to clinical data sets. This can be performed by a data processing system, such as computing system 1000 described below.

A multi-hit medium (MHM) treatment of the liver-only and adipose-only MPSs drives an insulin-resistant phenotype (metabolic dysfunction) and induces clinical hallmarks of NAFLD in the liver MPS. The MPSs are used to establish metabolically dysfunctional (MetS) liver and adipose MPSs and spheroids models. As components of a preclinical target discovery and drug development platform described herein, the MPSs 110, 120 are used to generate physiological relevant multi-scale (high-content) data by recapitulating disease microenvironment on liver-adipose axis. Multicellular spheroids function in a high-throughput screening capacity for treatment regimen optimization. The tissue-engineered MPSs 110, 120 mimic NAFLD-relevant cytoarchitecture and intercellular signaling microenvironments, including clinically relevant medium concentrations of nutrients (such as glucose, fructose, and FFAs) and signaling molecules (insulin and TNF-α). By establishing the relevant liver tissue (e.g., primary human hepatocytes, LSECs, Kupffer cells, and stellate cells) and visceral adipose tissue (e.g., primary human VAT) cytoarchitecture in recirculating MPSs 110, 120, clinically-observed phenotypic characteristics of MetS and NAFLD can be evaluated (e.g., either automatically or with human intervention) and compared to clinical phenotypes. The data generated by the MPSs 110, 120 is used to identify molecular “signatures” of NAFLD and/or MetS. Long term cultures (e.g., greater than 28 days old) of the tissue-engineered MPSs 110, 120 provide a platform to study NAFL-NASH pathophysiology, progression, and pharmacology.

As previously stated, the platforms, 100, 115 are used for target discovery and drug development. Each platform 100, 115 combines a tissue-engineered MPS “on-chip” model and a high-throughput spheroid screening model. Data generated by analyzing the behavior of the tissue of the platforms 100, 115 can be used to establish metabolically dysfunctional (MetS) MPSs and spheroid models for the liver and adipose tissue of a human. In an example, the primary human parenchymal and non-parenchymal liver cells can be sourced from a single donor for all aims and experiments to minimize the risk of adverse allograft interactions (e.g. Kupffer cell allo-antibodies). Metabolic dysfunction can be induced by culturing the MPSs 110, 120 in a defined, serum-free medium containing disease-relevant concentrations of glucose, fructose, insulin, FFAs, and TNF-α (multi-hit medium (MHM)) and then compared to MPSs cultured in a physiologically healthy medium (PHM). Metabolic dysfunction can be defined phenotypically as IR in each of liver and adipose MPSs 110, 120. Metabolic dysfunction can be confirmed using established functional assays of IR including insulin-stimulated glucose uptake and lipolysis. Following the establishment and characterization of the liver and adipose MPSs of metabolic dysfunction (MetS), the phenotypic, biomarker, and transcriptomic profiles are characterized and compared to the clinical NAFLD hallmarks (e.g., IR, steatosis, oxidative stress, steatohepatitis, and fibrosis, as previously described) to identify the pathophysiological relevance of the generated disease models.

The MetS-MPSs are used to evaluate both disease (and stage) specific biomarkers and also therapeutics targeting liver and adipose tissue alone and in combination and compared to CRISPR-mediated pathway perturbations for their effects on disease hallmarks. In addition to evaluating the abovementioned phenotypic NAFLD hallmarks, disease relevant biomarkers (e.g., aspartate aminotransferase (AST), alanine aminotransferase (ALT) and cytokeratin-18) and liver-adipose crosstalk dysfunction (e.g., hepatokines, adipokines, and cytokines) are evaluated, as subsequently described, and biomarker changes are associated with disease hallmark statuses (e.g., IR, steatosis, steatohepatitis).

Turning to FIG. 1C, the hardware platform 140, which represents either of platforms 100, 115, can be configured as follows. The platform 140 can include a top cover 142, a micropatterned microfluidic layer 144, and a bottom cover 146. The bottom cover 146 forms a base for the MPSs (e.g., MPSs 110, 120 of FIGS. 1A-1B) of the platform 140. The bottom cover 146 supports the various chambers and channels of the MPSs 110, 120 of the microfluidic layer 144. The bottom cover 146 is a solid platform that stabilizes the MPSs 110, 120 and provides a foundation for the MPSs.

The microfluidic layer 144 includes the chambers and channels of the MPSs 110, 120 for fluid flow through the system. The liver and/or adipose cells and tissues are hosted in these chambers and/or channels as needed to emulate organ functionality. The microfluidic layer 144 is patterned into a polymer material (e.g., a polycarbonate (PC)) as subsequently described. The microfluidic layer 144 is laminated between the bottom cover 146 and the top cover 142. The chip enables mono-cultures and co-cultures of various liver and adipose types in 2D and 3D. The cell source can be human primary cells, stem cells or cell lines. Alternatively, primary cells and/or immortalized cell line be used as sources for tissue in the MPSs. Generally, primary cells are cells that have been isolated and then used relatively quickly (e.g., immediately or after cryopreservation). Diseased cells representing one or more stages of NAFLD can be introduced into the cell chambers of the MPSs 110, 120.

The top cover 142 includes interconnecting tubes for each of the MPSs 110, 120. In an example, the tubes of the top cover 142 connect one MPS to another MPS for perfusion. The tubes enable fluid flow at the desired rate so that organ functionality is accurately emulated. Generally, the top cover 142, the bottom cover 146, and the microfluidic layer 144 are fabricated into thermoplastic materials. The fabrication can include one or more processes such as laser/plotter cutting, CNC machining, and microinjection molding. The MPS devices 110, 120 can be fabricated using thermoplastics such as polymethyl methacrylate (PMMA), polycarbonate (PC), polysulfone (PSU), or cyclic olefin copolymer (COC). In some implementations, the hardware platform 140 is PDMS-free to minimize undesirable non-specific adsorption of lipophilic molecules. The bottom cover 146 can be laminated with a gas permeable material to accurately reflect the incubator conditions. While three layers are shown in FIG. 1C, a fourth layer can be included. The fourth layer includes a membrane layer between the top cover 142 and the microfluidic layer 144. As shown in image 150, each platform 100 can include six MPSs, where each MPS can be perfused independently. In some implementations, the mixing and re-oxygenation chamber 108 or 130 can include an inlet (not shown) to supply oxygen to the media. Generally, oxygen can be depleted by the tissue in the MPS 110 or 120 (e.g., at a relatively fast rate for hepatocytes).

Turning to FIG. 1D, a graph 160 showing computational fluid dynamics (CFD) for a cell chamber (e.g., chamber 102 or 122a-b) is shown. The cell chambers can be designed using computational fluid dynamics (CFD) to provide physiologically relevant oxygen tension to the liver or adipose culture, while minimizing the shear stress (e.g., less than 0.05 dyne/cm²). The cell culture area is approximately 40 millimeters (mm) by 5 mm and holds 0.5-1 million cells. The laser-cut PC layers are laminated with an optimized bonding protocol (e.g., a thermal bonding protocol, a solvent-based bonding protocol, etc.). The chips are perfused with pneumatic pumps (or displacement pumps) with a wide range of flow rates (e.g., 0-100 mL/day). The MP S s 110, 120 are characterized for long-term culture biocompatibility (e.g., greater than 28 days) and validated using cell morphology, cell viability, and cytotoxicity assays (FIG. 1D).

FIG. 1E shows phase contrast images 170, 180 of adipocytes and hepatocytes cultured in PHM in the one-compartment MPS 110. To assist in visualizing the cells in the fluidic channels, various sizes of fluorescent dextran or dyes are introduced into the chips and visualized by confocal or epi-fluorescent microscopy.

The individual liver and adipose MPSs are established prior to combination into the interconnected configuration (e.g., on MPS 120). For the liver cell characterization, healthy patient, single donor, primary hepatocytes are seeded on ECM-coated substrates and evaluated over time (generally for more than 14 days). The primary hepatocytes are evaluated for their expressions and/or secretions of the phenotype-relevant proteins, including albumin, Aspartate Aminotransferase (AST), Alanine Aminotransferase (ALT), and cytokeratin-18. A similar phenotypic characterization is conducted for Liver sinusoidal endothelial cells (LSECs) such as Lymphatic vessel endothelial hyaluronan receptor 1 (LYVE1), stellate cells (e.g., glial fibrillary acidic protein (GFAP) and alpha smooth muscle actin (α-SMA)), and Kupffer cells (e.g., cell type CD68).

For adipocyte characterization, primary visceral pre-adipocytes are seeded on ECM-coated substrates, differentiated into adipocytes (e.g., for about 48 hours) following the vendor recommended protocol and evaluated for expression of the phenotype-relevant proteins. The phenotype-relevant proteins can include long-chain fatty acid transport protein 4 (FATP4), baseline lipolysis, and secretion of adipokines (such as leptin and adiponectin).

For a three dimensional (3D) liver MPS, the IR (MetS), and NAFLD phenotype determination is performed as follows. Hepatocytes and stellate cells are seeded into the ECM-coated liver compartment of the MPS device. Following a 24 hour equilibration period, an extracellular matrix (ECM) hydrogel topcoat containing LSECs and Kupffer cells is seeded into the liver compartment of the MPS device (e.g., MPSs 110, 120). After an equilibration and maturation period (e.g., 3 days), the liver MPS is switched either to MHM or PHM and monitored for metabolic dysfunction for up to 21 days. The liver MPS is evaluated for induction of the IR phenotype by monitoring their insulin-stimulated glucose uptake (e.g., using a glucose uptake-glo assay from Promega) compared to healthy controls. Both hepatokine (e.g., fetuin-A and fibroblast growth factor 21 (FGF21)) and cytokine (e.g., IL-1β, IL-6, TNF-α) concentrations and their correlation to the IR phenotype are determined. The NAFLD phenotype is evaluated by evaluating a degree of steatosis and fibrosis.

For a 3D adipose MPS, the IR (MetS) phenotype determination is performed as follows. Pre-adipocytes are seeded in an ECM hydrogel into the adipose tissue compartment and differentiated into adipocytes (e.g., for about 48 hours) following the vendor recommended protocol. After an equilibration and maturation period (about 3 days), the adipose MPS is switched either to MHM or PHM and monitored for metabolic dysfunction for up to 21 days. Adipocytes are evaluated for induction of the IR phenotype by monitoring lipolysis levels (e.g., using a glycerol-glo assay from Promega) compared to healthy controls. Adipokine (e.g., leptin and adiponectin) and cytokine (e.g., IL-1β, IL-6, TNF-α) concentrations and their correlations to the IR phenotype are determined.

As a high-throughput screening platform, liver and adipocyte spheroid cultures (for 3D spheroid models) are established following validated protocols and cultured in MHM or PHM following the same timeline as used for the liver and adipose MPSs. Clinical hallmarks of NAFLD (e.g., IR, steatosis, and dysregulated adipokine signaling) are observed as early as day 7. Hallmarks of progression towards NASH (e.g., worsening steatosis and steatohepatitis IL-6, and TNF-α)) are observed as late as day 21.

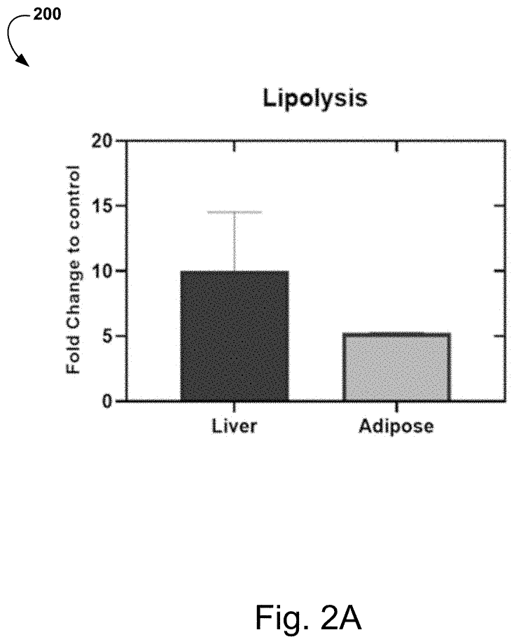

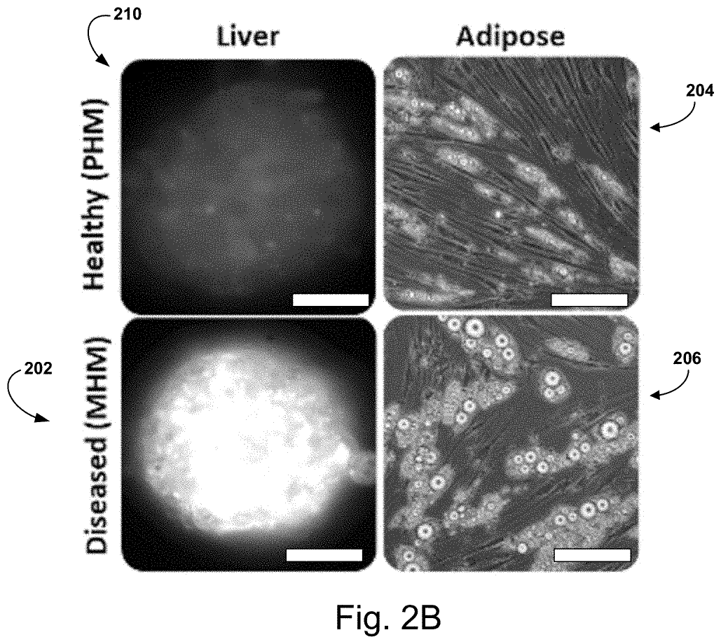

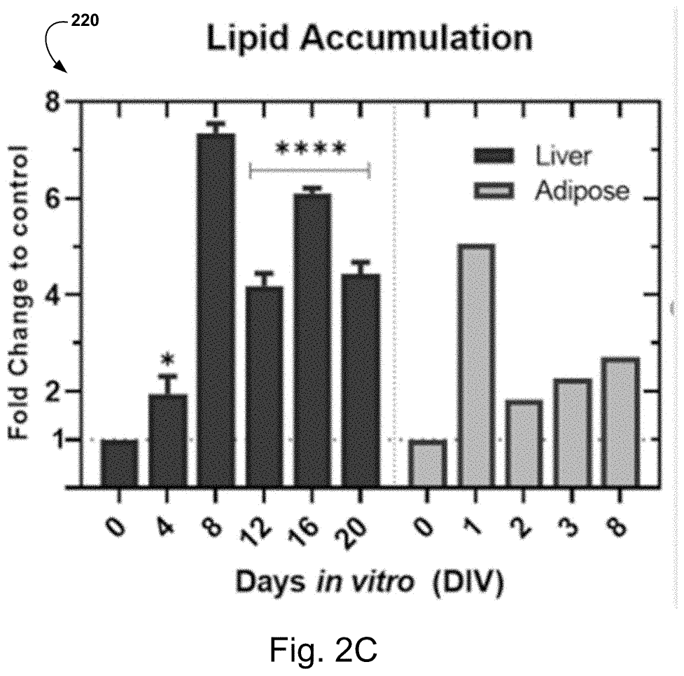

Turning to FIGS. 2A-2C, example data representing a result of a process for establishing metabolically dysfunctional MPSs (e.g., liver and adipose MPSs 110, 120 of FIGS. 1A-1E) to demonstrate emulated metabolic syndrome and NAFLD in accordance with the previously described processes are shown. Hallmarks of NAFLD emerge in liver and adipose tissue independently after treatment with NAFLD-induction media (MHM). In FIG. 2A, graph 200 shows an increased lipolysis in cells grown in MHM (diseased medium), determined by extracellular glycerol production, indicates an insulin resistant (IR) state. In FIG. 2B, images 210, 202, 204, 206 show increased intracellular lipids (TAG) visualized using AdipoRED fluorescent staining (liver spheroids 210, 202) or phase contrast (adipose 204, 206), confirming steatosis, a major hallmark of NAFL. The scale bar is 100 micrometers. FIG. 2C shows a graph 220 that shows that the disease state is further confirmed by the progressive accumulation of intracellular lipids (TAG), quantified from fluorescent and brightfield images. (*p<0.05, ****p<0.0001).

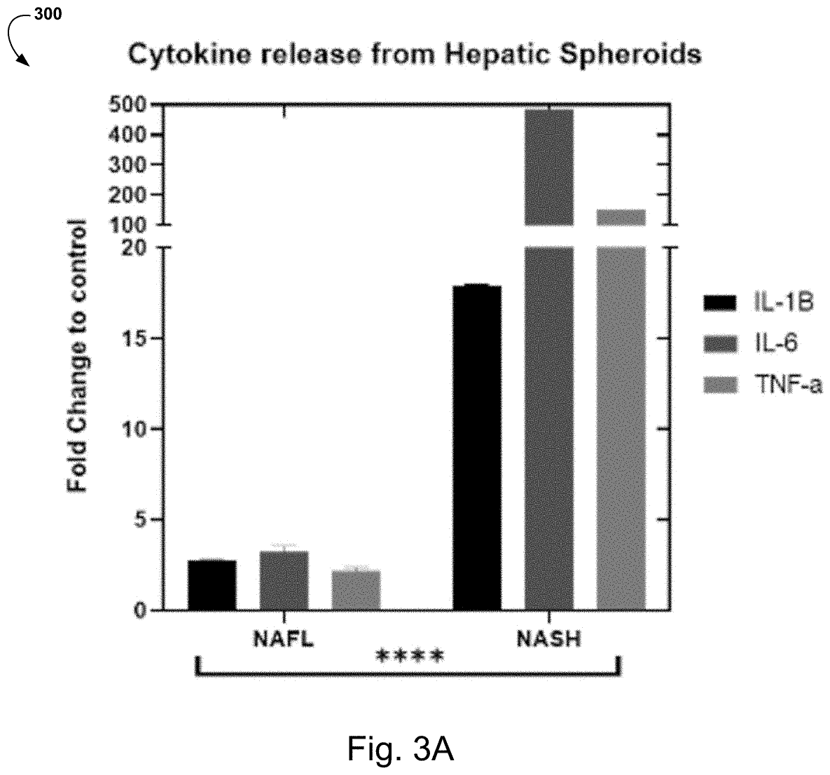

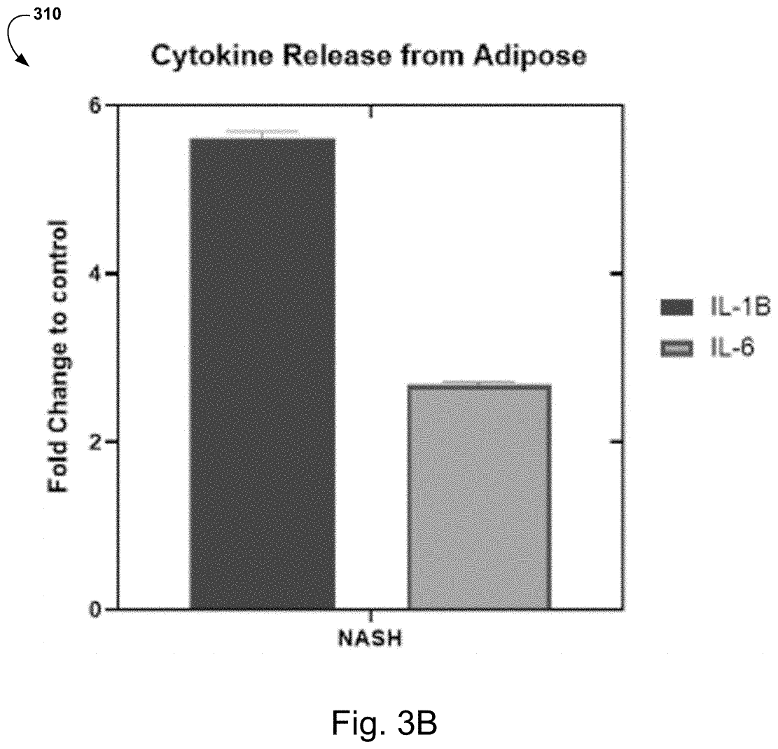

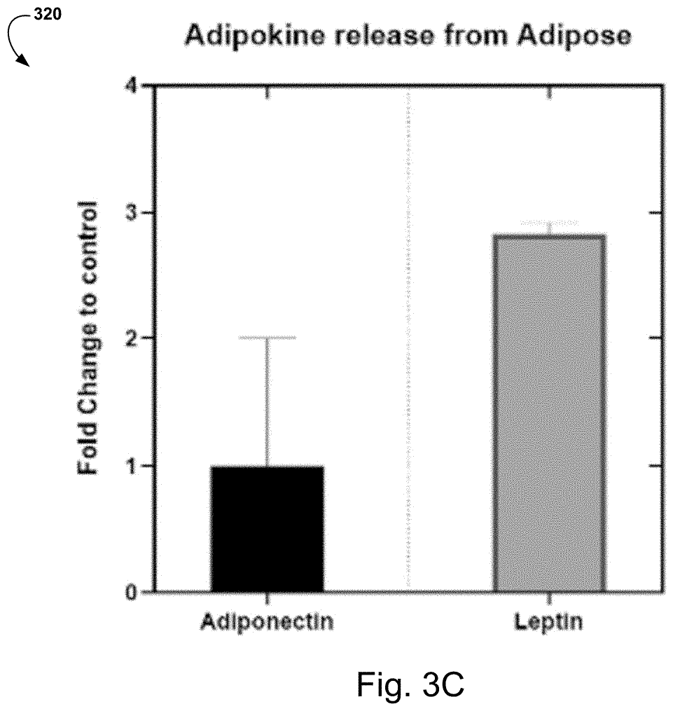

FIGS. 3A-3C show example data indicating that the elevated cytokine release from hepatic spheroids and adipocytes mark the transition to NASH. In FIG. 3A, graph 300 shows that, relative to PHM-treated cells, concentrations of inflammatory cytokines (TNF-α, IL-6, IL-1β) are significantly elevated in NASH (MHM treated) spheroids. Moreover, cytokines released from NAFL (fatty acid-only treated) spheroids indicate IR and steatosis alone induce mild inflammation and prime the tissue for the transition to NASH (****p<0.0001, n=4). In FIG. 3B, graph 310 shows that cytokine release from adipose is also markedly elevated after MHM-treatment (n=2). In FIG. 3C, graph 320 shows that increased recirculating adipokine concentrations in MHM-cultured adipocytes are further evidence of MetS transition to NASH (n=2).

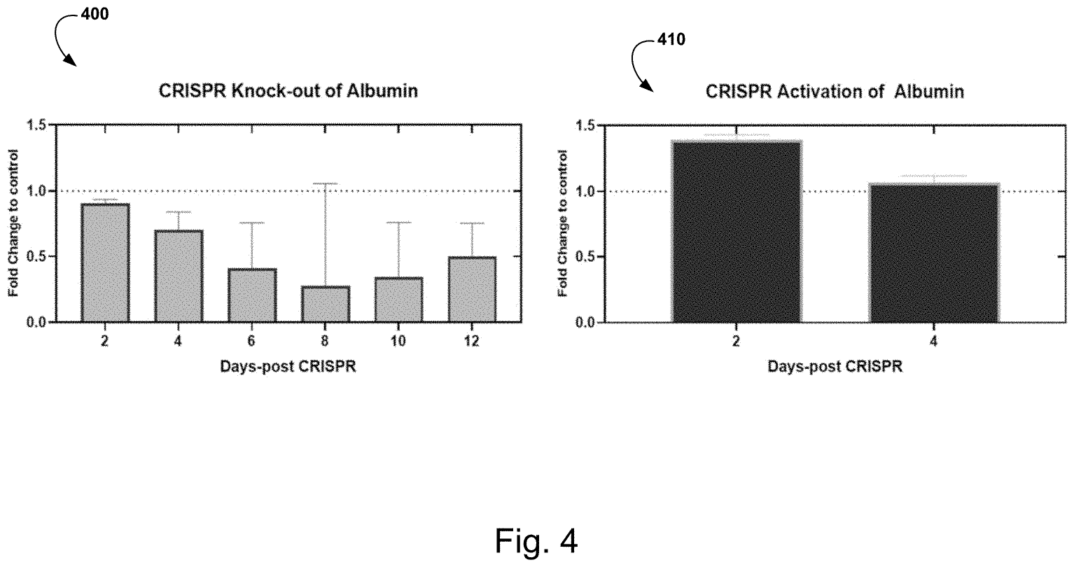

FIG. 4 illustrates example graphs 400, 410 that show CRISPR-mediated ALB (albumin) gene perturbations in hepatic spheroids. Graph 500 shows a progressive reduction in medium albumin concentration indicates knock-out (KO) of ALB gene. Graph 410 shows a 48 h hour increase in medium albumin concentration indicates transient gene activation.

Graphs 400, 410 show results of using the MPSs 110, 120 to assess the clinical relevance of MPS pathophysiology using single-MPS transcriptomics and known disease modifying perturbations, compared with clinical findings. A consideration in developing a complex human in vitro model is the recapitulation and conservation of physiological gene expression and regulation for tissue functionality. This is essential for thorough understanding of disease inducing mechanisms and for the potential use of these complex in vitro models as target discovery platforms. The MPSs 110 are used to generate transcriptomics data for healthy and NAFLD phenotypes. For example, RNA-Seq (mRNA expression) experiments are performed producing 20-30M read pairs per sample, which will provide sufficient sequencing depth for differential expression studies. The RNA-Seq analysis workflow starts with processing of raw reads to quantify transcript abundances using a pseudo-alignment-based approach. Gene counts are then used for differential expression analysis (DEA), followed by a functional analysis. DEA and functional analysis results demonstrate the disease phenotypes in the single-MPS 110, highlighting the disease associated alterations in gene expression and biological pathways.

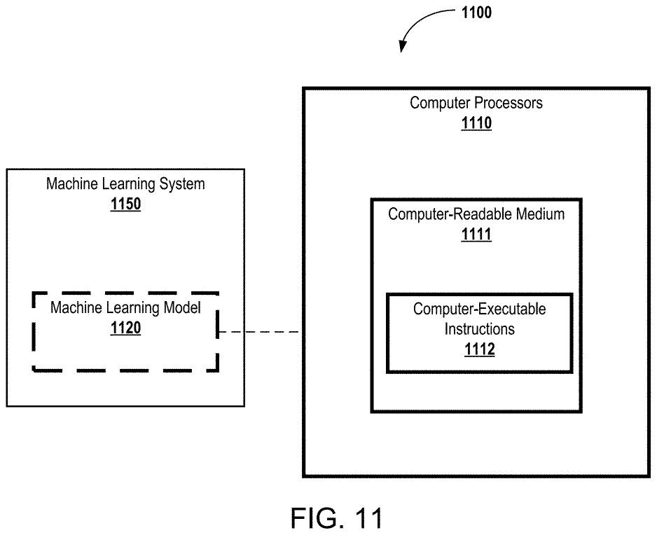

In silico evaluation is performed to demonstrate clinical relevance, such as using the computing system 1100 subsequently described in relation to FIG. 11. The physiological relevance of the single-MPS 110 and induced NAFLD phenotypes is shown using clinical datasets comprising gene expression data measured in human tissue/patient biopsy samples. For this purpose, data such as from the National Center for Biotechnology Information (NCBI) Gene Expression Omnibus (GEO) repository can be used for human datasets and develop MPS-to-human translation model, a framework of translational machine learning (ML) models. GEO archives a wide collection of curated sequencing data from multiple platforms (and arrays), such as genomics and transcriptomics, for users to query, review and download studies. MPS-to-human translational effort can stage MPS phenotypes for better stratification and accurate therapeutic intervention. Using GEO datasets annotated with clinical diagnostic information, the MPS-to-human model is trained and validated to predict a NAFLD activity score (NAS) or the disease stage from gene expression data. Clinical diagnostic information is used for calculation of area under receiver operating characteristic (ROC) curve (AUC) and root-mean-square-error (RMSE), respectively, to evaluate the ML models and prediction results. Validated ML models are used for the translation of MPS gene expression datasets to clinical phenotypes. MPS-to-human methodology can be used to predict the disease stage and severity of different MPS phenotypes representing subpopulations.

Experimental target perturbation is performed to rapidly screen drugs and drug combinations and optimize dosing regimens (and CRISPR sgRNAs) for evaluation in the MPS models. The healthy and diseased liver and adipose spheroid cultures are established as previously described. Following NAFLD induction, spheroid cultures are treated with small molecules or CRISPR short guide RNAs (sgRNAs) that modulate the activity or expression of disease-relevant signaling pathways (e.g. FXR, PPARα, PPARβ/δ, PPARγ, and CCR2-5). Generally, liver spheroid gene expression can be perturbed using CRISPR-KO and CRIPSR-activation, as shown in graphs 400, 410. Specifically, CRISPR-KO of the ALB gene reduced medium albumin concentration by ˜75%, whereas CRIPSR-activation increased medium albumin concentration ˜50%.

To perform target perturbation, small molecules (e.g. rosiglitazone (PPARγ), elafibranor (PPARα and PPARβ/δ), obeticholic acid (OCA) (FXR), and cenicriviroc (CCR2-5)) are tested at five concentrations spanning a nanomolar to millimolar range to develop a dose-response relationship. The concentration of the drug in the medium is maintained throughout the treatment period using a repeat dosing strategy applied during media changes. CRISPR-mediated pathway modulation (CRISPR activation or inactivation) is maintained throughout the treatment period by using lentiviral-mediated sgRNA delivery methods. The most efficacious small molecule or CRISPR treatment regimen (or treatment combination) is determined by evaluating the reduction in disease state (steatosis and/or fibrosis) compared to untreated diseased spheroids. Subsequently, the healthy and diseased liver and adipose MPSs are treated with the optimized treatment protocol identified using the high-throughput screening spheroid models. The treatment efficacy in the liver and adipose MPSs is determined using phenotypic (e.g., IR, steatosis, and fibrosis) and biomarker (e.g., apoptosis, hepatokine, adipokine, cytokine) readouts as previously described.

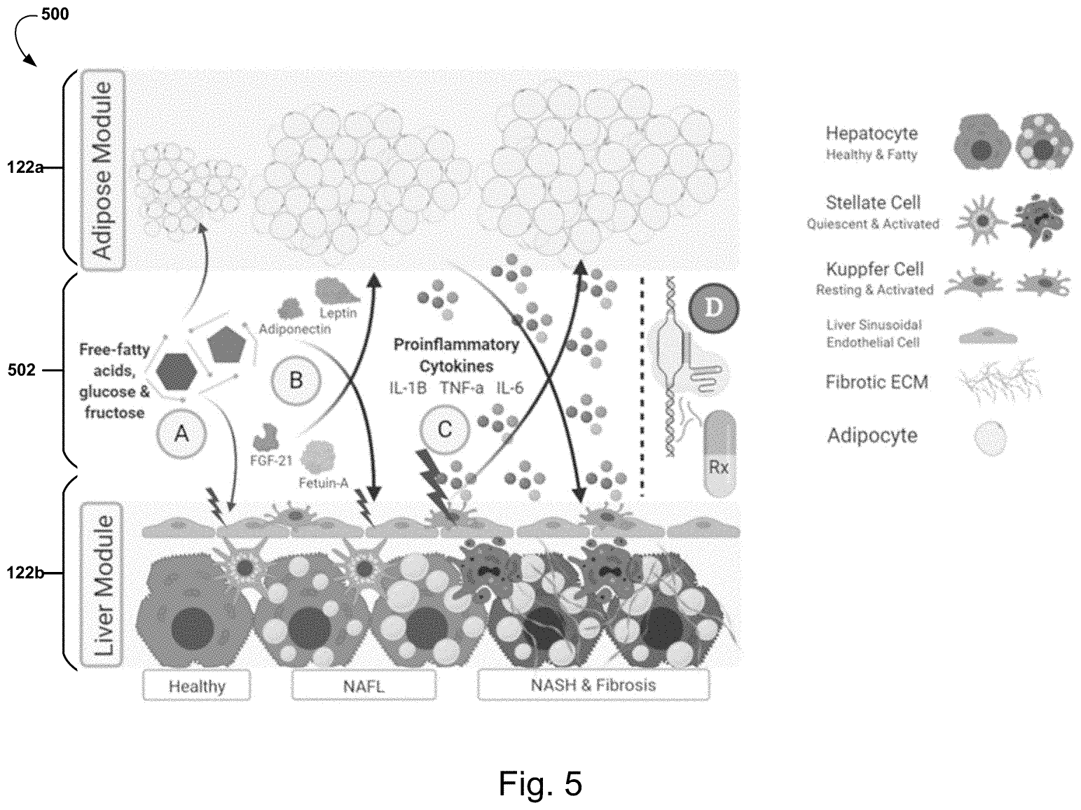

FIG. 5 shows liver-adipose crosstalk 500 in multi-compartment MPS 120. The NAFLD-associated multi-hit pathology is represented by excess circulating nutrients to drive IR and steatosis, labeled A. The NAFLD-associated multi-hit pathology is represented by dysregulated adipokine and hepatokine crosstalk signaling, labeled B. The NAFLD-associated multi-hit pathology is represented by proinflammatory cytokine stimulus and response driving progression towards NASH, labeled C. The NAFLD-associated multi-hit pathology is represented by combinatorial therapeutic and disease-relevant CRISPR-mediated target perturbation strategy for amelioration of NAFLD pathology, labeled D.

The preclinical target discovery and drug development platform is improved from single MPS 110 by combining the tissue-engineered liver and adipose MPSs into a single liver-adipose “crosstalk” MPS 120, as previously described in relation to FIG. 1B. MPS 120 device is used to investigate the role of the liver-adipose axis and crosstalk signaling (e.g., hepatokines, adipokines, and cytokines) on NAFLD severity and progression. The crosstalk 502 is illustrated in mechanisms A-D of FIG. 5. Additionally, intracellular metabolomics are added to our transcriptomic data, significantly improving the mechanistic systems biology element of the preclinical target discovery platform 115. Specifically, multi-omics data (e.g., transcriptomics and metabolomics) are generated using the clinically relevant disease phenotypes. An integrated network model NAFLD-Net is built to identify potential disease modifying targets in the diseased states.

FIG. 6A shows an example workflow 600 for a preclinical target discovery. There are consecutive steps between the liver-adipose crosstalk MPS and the computational framework NAFLD-Net. Liver-adipose crosstalk MPS is used to generate (602) multi-omics data to build NAFLD-Net 604, which consists of context-specific genome scale metabolic models 606 (GEMs). Therapeutic targets 608 identified by NAFLD-Net are prioritized (610) followed by high-throughput screening 612 using spheroid cultures. The resulting target shortlist 614 is validated (616) using the liver-adipose MPS 120.

Generally, heterogeneous risk profiles and variability in response to therapeutic interventions are challenges in drug development for NAFLD. Consequently, the liver-adipose MPS 120 and NAFLD-Net 604 computational model are applied to two stratified patient subpopulations based on differential biological mechanisms including a high-risk genetic single nucleotide polymorphism (SNP) (PNPLA3 SNP rs738409 (I148M) and pre- vs. post-menopausal (sex hormone-based). These are “population specific MPSs.”A stratified medicine approach generates phenotypic, transcriptomic, and metabolomic datasets establishing a deep molecular characterization of each subpopulation.

Currently, there are few, if any, human cell-based, in vitro models to study patient subpopulations. Furthermore, most medicines are currently developed and prescribed empirically, with few effective strategies to match potential patients with therapies most likely to be effective and safe. The platform 115 is used to identify unique, population-stratified therapeutic targets of NAFLD by characterizing and validating NAFLD liver-adipose crosstalk MPSs 120 based on clinically relevant disease subpopulations. The identified disease modifying targets are validated using functional genomics tools (e.g. CRISPR) to assess the disease modifying potential of the target pathways by evaluating changes in disease phenotypes (such as fibrosis, lipid accumulation, cytokine release). A liver-adipose axis MPS is established and a data-informed “stratified medicine” liver-adipose MPS are formed for identification of dysregulated pathways and disease modifying targets, and drug discovery and development (DDD) for NAFLD.

The liver-adipose crosstalk MPS 120 is used to demonstrate NAFL-NASH disease progression for preclinical target discovery and stratified medicine applications. Generally, a multi-hit medium (MHM) treatment of the liver-adipose MPS progressively induces the clinical hallmarks of NAFL (e.g., IR and steatosis) and NASH (e.g., steatohepatitis and fibrosis). The metabolically dysfunctional liver and adipose MPSs are integrated into a single liver-adipose crosstalk MPS 120, as previously described. Clinical data indicates that metabolically dysfunctional adipose tissue is the source of FFAs that drive liver steatosis. Additionally, dysfunctional adipokine signaling is known to exacerbate liver dysfunction. By combining the recirculating adipose tissue and liver MPSs 122a-b, the clinically-observed phenotypic characteristics of NAFLD and the disease-relevant crosstalk that contributes NAFL and NASH are evaluated using a single device. Consequently, the MPS, in combination with multi-omics datasets, can be used to identify molecular “signatures” of disease and potential disease modifying targets and combination therapies on adipose-liver axis. To understand the complexity of NAFLD pathophysiology and the molecular interrelationships, an integrative systems biology approach is taken by combining multi-omics data sets. This stable, tissue-engineered, crosstalk MPS 120 provides a platform to study NAFL-NASH pathophysiology, progression, and pharmacology.

A tissue-engineered liver-adipose MPS 120 is established and cultured in MEM or PHM. NAFLD phenotypic, biomarker, transcriptomic, and intracellular metabolomic profiles are characterized and compared to the clinical disease hallmarks (e.g., IR, steatosis, oxidative stress, steatohepatitis, and fibrosis) to identify the pathophysiological relevance of the model. The multilayered -omics systems biology approach facilitates a deeper understanding of the biological/pathological interconnections driving NAFLD. Using the liver-adipose crosstalk MPS 120, small molecule therapeutics targeting liver and adipose tissue are evaluated alone and in combination and compared to CRISPR-mediated pathway perturbations for their effects on disease hallmarks. In these experiments, NAFLD-relevant phenotypic biomarkers (e.g. steatosis, steatohepatitis, and fibrosis), clinically-relevant biomarker (aspartate aminotransferase (AST), alanine aminotransferase (ALT) and cytokeratin-18) and liver-adipose crosstalk biomarkers (hepatokines, adipokines, and cytokines) are evaluated.

Generally, liver-adipose crosstalk MPS 120 validation, establishment, and characterization are performed as follows. To create physiologically relevant adipose-liver microenvironment and crosstalk, the individual liver and adipose MPSs are integrated into one recirculating chip with two compartments 122a-b connected by connecting outlets of each MPS into a mixing compartment 130 as previously described in relation to FIG. 1B. Generally, the integrated chip compartment sizes (cell culture surface area, seeding density and volume) are informed with the multi-functional mechanistic scaling methodology described previously. CFD modeling is used to design the fluidic channels based on the computational flow characteristics. The concentration gradient and distribution of molecules secreted from cells or molecules/drugs introduced in the system are simulated. Pre-adipocytes are seeded using a 3D hydrogel into the adipose tissue compartment and differentiated into adipocytes (e.g., for 48 hours) following the vendor recommended protocol. Concurrently, hepatocytes and stellate cells are seeded into the ECM-coated liver compartment of the MPS device.

Following a 24 hour equilibration period, an ECM hydrogel topcoat containing LSECs and Kupffer cells is seeded into the liver compartment of the MPS device 120. After an equilibration and maturation period (e.g., 3 days), the liver-adipose MPSs 120 are switched to either MHM or PHM and monitored for disease progression for up to 21 days. During the 21-day experiment, three periodic MPS takedowns are conducted to evaluate discreet disease phases (NAFL, NASH-fibrosis, NASH+fibrosis) and disease progression via histological analysis of the liver (steatosis and fibrosis) and adipose modules (IR and steatosis). Additionally, RNA or intracellular metabolite extraction from the liver and adipose modules is conducted to determine the transcriptomic and metabolomic profiles associated with NAFL-NASH disease progression. Cell culture medium is reserved for analysis of apoptosis, hepatokine, adipokine, and cytokine (crosstalk biomarker molecules) concentrations.

To perform in silico evaluation of clinical relevance, the liver-adipose MPS transcriptomics for healthy and NAFLD phenotypes are used for differential expression and functional analysis. Single-MPS 110 results (previously described) are compared to liver-adipose crosstalk MPS results to demonstrate the heterogeneity of disease phenotypes, highlighting the contribution of liver-adipose crosstalk to disease associated alterations in gene expression and biological pathways. Clinical relevance of the liver-adipose crosstalk MPS 120 and induced NAFLD phenotypes is evaluated using MPS-to-human framework to predict the disease stage and severity in liver-adipose crosstalk MPS 120.

FIG. 6B shows a multi-omics analysis 650 and pathway-based integration. Graph 652 shows a pathway enrichment analysis of metabolomics. Data 654 shows a gene ontology enrichment analysis of transcriptomics. Chart 656 shows an over-representation analysis of integrated -omics datasets for comparison of MPS-cultured and suspension-cultured PHHs. In chart 656, statistical significance of metabolomics (qm), transcriptomics (qt), and integrated omics (qjoint) are shown as bars in the middle, right, and left, respectively.

The models of FIGS. 6A-6B result from generated multi-omics data using the liver-adipose MPS 120 and developed into in silico target discovery framework NAFLD-Net to identify molecular pathophysiological networks and therapeutic targets. Multi-layer omics and systems biology approaches allow mapping high-content and high-throughput data and characterize patient-specific disease phenotypes to deliver precision medicine solutions. Integration of omics layers increases the predictive power while providing a mechanistic link between the genotype and observed physiological state. Transcriptomics provides global gene expression profile and how it changes under conditions (healthy vs. diseased) to inform on disease mechanisms, whereas metabolomics is the closest layer to the phenotype, and provides signatures of biochemical activity. Transcriptomics, extracellular metabolomics, and intracellular metabolomics datasets are generated using the liver and adipose tissues collected from the crosstalk MPS for healthy and NAFLD phenotypes (e.g., cultured in MHM or PHM). RNA-Seq (mRNA expression) experiments are performed using both liver and adipose samples as previously described. Intracellular metabolome experiments using the liver and adipose samples are conducted. Global profiling (e.g., more than 1200 metabolites) are attained by CE-MS and LC-MS platforms to capture both hydrophilic and hydrophobic metabolites to cover fatty acids, steroids, and other lipids in addition to sugars, amino acids, nucleotides, and other ionic metabolites.

Pathway-based integration of transcriptomics and metabolomics are applied as implemented in Integrated Molecular Pathway Level Analysis (IMPaLA) and commercial Ingenuity Pathway Analysis (IPA) for joint over-representation and enrichment analysis on canonical pathways. This multi-layer omics approach is more informative and powerful than single-omics to characterize disease progression and to compare the induced NAFLD phenotypes. The MPS-cultured and suspension-cultured primary human hepatocytes (PHHs) are characterized and compared using single-omics (metabolomics, transcriptomics) and their pathway-based integration, as previously described.

For target discovery efforts, the NAFLD-Net model 604, a computational framework of multi-tissue (liver-adipose) genome-scale metabolic models (GEMs), is used to study NAFLD pathophysiology. The NAFLD-Net 604 model stratifies distinct biological/clinical profiles by linking genotypes to phenotypes through gene-protein-reaction associations. Tissue-specific models for liver and adipose are reconstructed from global human metabolic network models such as Human Metabolic Reaction (HMR 2.0) or Recon 3D using tissue-specificity information from UniProt, Human Protein Atlas, and MPS transcriptomics data generated by MPSs 110, 120. Validated and functional liver- and adipose-specific GEMs are integrated through a media (common pool) compartment to construct the liver-adipose GEM representing the baseline (healthy) metabolic state. This network model can be used as a scaffold to map omics data, to run simulations for different metabolic states (such as fasting vs. fed states), and to generate hypotheses on disease associated mechanisms for target discovery.

MPS transcriptomics datasets are mapped on the liver-adipose GEM to construct the context-specific multi-tissue networks (e.g., as shown in process 600). Metabolic states for healthy, steatosis (NAFL), steatohepatitis (early NASH), and NASH with fibrosis phenotypes are compared to elucidate disease progression and differential metabolic reactions. Intracellular metabolomics datasets inform on the phenotypic outcome and disease related alterations in reaction activity and are used to validate the predicted differential reactions by NAFLD-Net. Context-specific GEMs constructed in NAFLD-Net framework and omics data mapping generate therapeutic target lists based on differential reactions. These therapeutic targets include mRNAs and proteins, which are indirectly or directly associated with metabolic reactions, as well as metabolites to be supplemented due to disease-related mechanistic deficiency. The long lists are rank-ordered to prioritize the targets with respect to several criteria such as druggability, therapeutic evidence in clinical data, tissue specificity, safety/toxicity information, and novelty. Efficacy, safety, and novelty information from existing databases are used for target prioritization. As a result of prioritization, therapeutic target shortlists are achieved and experimentally validated using spheroid cultures and the liver-adipose MPS.

The disease modifying potential of targets is evaluated using one or both of large and small molecules and functional genomics tools, such as CRISPR. For example, large molecules can include ligand antagonists, Humira (adalimumab) blocks, and TNF-α. Healthy and NAFLD liver-adipose MPSs are established previously described. Following disease induction, the liver-adipose MPSs are treated with small molecules (alone and in combination) or CRISPR short guide RNAs (sgRNAs) that modulate the activity or expression of disease-relevant signaling pathways (e.g. CCR2-5, FXR, PPARγ, PPARβ/δ, and PPARγ) as previously described in relation to FIG. 4. The dosing regimen are optimized using the efficacy data generated using the liver and adipose spheroids previously described. The disease-modifying potential of the therapeutic treatment regimens in the liver-adipose crosstalk MPS 120 are determined using phenotypic (e.g., IR, steatosis, steatohepatitis, and fibrosis) and biomarker (apoptosis, hepatokine, adipokine, cytokine) readouts.

These experiments can demonstrate the pathophysiological relevance of the liver-adipose crosstalk MPS 120. In the liver-adipose MPS 120, MHM induces a NAFLD phenotype in the liver (e.g., oxidative stress, steatosis, and fibrosis) and adipose tissue (e.g., steatosis) compared to MPSs cultured in healthy medium. Clinical biomarkers of NAFLD (e.g., AST, ALT, cytokeratin-18) are elevated in diseased systems compared to healthy systems. Additionally, crosstalk signaling molecule concentrations are dysregulated in diseased systems compared to healthy systems and those changes trend with clinical patient findings (e.g. IL-6↑, IL-1β↑, leptin ↑, adiponectin ↓, fetuin A ↑, FGF21↑) (16, 18, 36). The NAFLD phenotype is more severe in the liver-adipose MPS compared to the liver-only MPS due to adipose tissue pathophysiology and dysregulated crosstalk signaling. Healthy and disease phenotypes have differential gene expression and metabolome signatures, corresponding to multi-hits in NAFLD progression and compare to the clinical findings. Genome-scale metabolic models (GEMs) and pathway-based integration of omics datasets are more predictive compared to single-omics analysis by decreasing the number of false positives in differential molecular signatures. Small molecule (e.g., elafibranor, OCA, rosiglitazone, cenicriviroc) and/or CRISPR-mediated transcription factor perturbations of disease relevant pathways reduce the severity of the steatosis, fibrosis, and insulin resistance in the liver-adipose crosstalk MPSs.

The disease relevant molecule (stellate cell activating) transforming growth factor-β1 (TGF-β1) can be added to the medium if fibrosis development is insufficient during the experimental period. While the NAFLD-Net framework 604 provides a complete multi-tissue metabolic network (gene-protein-reaction associations) as a scaffold for mapping datasets, multi-tissue GEMs do not account for gene regulatory mechanisms. To replace a dynamic in silico disease model to recapitulate transcriptional and post-translational regulations, the model is retained for performing experiments and generating data for epigenomics and object-oriented in silico liver disease models are available for adaptation.

In an aspect, the data generated by the MPS device 120 can be used to investigate the NAFLD-NASH pathophysiological profile differences associated with the PNPLA3 SNP rs738409 (I148M). The patatin-like phospholipase domain-containing 3 (PNPLA3) gene encodes a transmembrane protein expressed in hepatocytes and stellate cells. The PNPLA3 protein is a triacylglycerol lipase that hydrolyzes triglycerides in hepatocytes and retinyl esters in stellate cells (68). The genetic polymorphism rs738409 C>G in the PNPLA3 gene leads to an amino acid substitution of isoleucine (I) to methionine (M) at position 148 (I148M). The mutated amino acid is located in the enzyme catalytic site and leads to loss of enzyme function. The effects of the PNPLA3 rs738409 polymorphism on the phenotypic, transcriptomic, and metabolomic disease progression profiles are evaluated in the liver-adipose MPS. These data sets can be analyzed using the NAFLD-Net computational model to identify novel gene expression signatures associated with NAFLD. The identified pathways can be perturbed using small molecules and functional genomics approaches to understand their role in disease etiology and progression. A PNPLA3 rs738409 polymorphism patient subpopulation stratified medicine MPS can be developed for use in novel pathway identification and preclinical drug discovery and development.

The PNPLA3 rs738409 polymorphism (M148) drive liver (directly) and adipose (indirectly, e.g. crosstalk signaling) dysfunction, and therefore, the NAFL-NASH disease profile in a mechanism-specific way compared to MPSs containing a healthy liver. Further, there are underlying disease-driving mechanisms are sufficiently different as to identifiable by multi-omics analysis, and therefore, benefit from stratification and tailored therapeutic intervention strategies.

The genetic polymorphism (rs738409 C>G) in the PNPLA3 gene is the strongest predictor of NAFLD (odds ratio=3.12, P<0.001) among the independent genetic risk factors of disease. Furthermore, patients carrying the rs738409 genetic variant exhibit increased hepatic steatosis, reduced VLDL secretion, and increased fibrosis and risk for disease progression to hepatocellular carcinoma. Establishing a PNPLA3 rs738409 stratified medicine model of NAFLD lead to improved success for new chemical entities during the preclinical stage of drug development.

To evaluate the role of the PNPLA3 (rs738409 C>G) SNP (I148M) on NAFLD progression, bio-banked lots of hepatocytes and stellate cells are genotyped using a probe-based qPCR genotyping assay. This rapid, robust detection method allows for biallelic polymorphism detection using two fluorescent probes in a single assay. PNPLA3 (rs738409 C>G) mutant hepatocytes and stellate cells are used in combination with LSECs and Kupffer cells to establish the “mutant” liver as previously described. The “healthy” liver is composed entirely of WT cells with matching biometric data to minimize the risk of donor variability. As an alternative, the PNPLA3 polymorphic hepatocytes and stellate cells are corrected to the WT allele using CRIPSR/Cas9 targeted gene editing. Following MPS establishment, multi-omics data are generated and potential therapeutic targets are identified. Next, the disease-relevant targets of interest are perturbed using small molecule and CRISPR sgRNAs in combination (and singly as a control) to evaluate the disease modifying potential combinatorial therapeutic treatment regimens for NAFLD.

In an aspect, a PNPLA3 (I148M) mutant liver-adipose crosstalk MPS 120 is characterized and demonstrate NAFL-NASH disease progression. Healthy and PNPLA3 mutant (polymorphic hepatocytes and stellate cells) liver-adipose crosstalk MPSs are established as previously described. After an equilibration and maturation period (e.g., 3 days), liver-adipose MPSs 120 are switched to either MHM or PHM and monitored for disease progression for up to 21 days. During the 21-day experiment, three periodic MPS takedowns are conducted to evaluate disease progression via phenotypic (e.g., histological and biomarker) analysis of the liver (e.g., steatosis and fibrosis) and adipose modules (e.g., steatosis). Additionally, RNA or intracellular metabolite extraction from the liver and adipose modules are conducted to determine the transcriptomic and metabolomic profiles associated with NAFL-NASH disease progression. At each takedown time-point, cell culture medium are reserved for recirculating biomarker analysis including hepatokine, adipokine, and cytokine (crosstalk molecules) concentrations as previously described and shown in the data of FIGS. 3A-3C.

In an aspect, multi-omics data are generated using the liver-adipose MPS 120 and NAFLD-Net for PNPLA3 polymorphism-based stratified medicine and identification of therapeutic targets is implemented. Transcriptomics and intracellular metabolomics datasets are generated from PNPLA3 WT (I148) and SNP rs738409 mutant (M148) MPSs for healthy and NAFLD phenotypes (e.g., cultured in MHM or PHM), and analyze the datasets as previously described. Using a multi-tissue (liver-adipose) GEM framework NAFLD-Net 604, context-specific, multi-tissue networks are constructed as described in relation to FIG. 6A. This framework is used for both phenotypes (WT and mutant) to assess the association of the PNPLA3 polymorphism with NAFLD progression and severity. The therapeutic target lists are generated based on NAFLD-Net results and the targets are prioritized as previously described. Prioritized targets are experimentally validated using CRISPR-mediated perturbation in spheroid cultures (e.g., described in relation to FIG. 4) and the liver-adipose crosstalk MPS 120.

In an aspect, the disease modifying potential of PNPLA3 mutant subpopulation-specific targets is evaluated using small molecules and functional genomics (e.g., CRISPR activation or inhibition). PNPLA3 rs738409 (M148) liver spheroids cultured in PHM (healthy) and MHM (diseased) are established as previously and used to rapidly screen disease-modifying target pairs using small molecule+CRISPR sgRNA combinations. Specifically, the top-ranked disease modifying targets identified using the NAFLD-Net are investigated in combination with the most clinically advanced small molecules (e.g. elafibranor+CRISPR sgRNA targeting the top-ranked inflammatory signaling pathway) to evaluate the synergistic effects on disease phenotype.

Additionally, the liver spheroids are treated with combinations of known small molecules (e.g. cenicriviroc, rosiglitazone, elafibranor, and OCA) that modulate the activity or expression of disease-relevant signaling pathways (e.g. CCR2-5, FXR, PPARγ, PPARβ/δ, and PPARγ) as previously described. In these screening experiments, the ability of the disease-modifying combinations to rescue the NAFLD phenotype are investigated using a dose-response approach. The most efficacious small molecule and/or CRISPR treatment regimen (disease-modifying target combination) are determined by evaluating the reduction in disease state (steatosis, fibrosis, inflammation, etc.) compared to untreated PNPLA3 mutant (M148) diseased spheroids. These disease modifying targets are evaluated using the PNPLA3 polymorphic crosstalk MPS to evaluate their suitability as candidate drug development targets. Accordingly, PNPLA3 rs738409 (M148) healthy and NAFLD liver-adipose MPSs are established as previously described.

Following disease induction, the PNPLA3 mutant liver-adipose MPSs are treated with the most efficacious combinatorial treatment regimen (e.g., small molecule+CRISPR sgRNA) identified using the high-throughput screening spheroid model. The disease modifying potential of the therapeutic treatment regimens in the mutant liver-adipose crosstalk MPSs 120 are determined using phenotypic (e.g., IR, steatosis, and fibrosis) and biomarker (e.g., apoptosis, hepatokine, adipokine, cytokine) readouts as previously described.

These experiments demonstrate the pathophysiological relevance of the PNPLA3 (rs738409 C>G) polymorphism stratified medicine platform including data driven target identification and perturbation. Baseline steatosis and fibrosis are increased in PNPLA3 mutant (M148) liver-adipose MPS, without IR, compared to the non-mutant (I148) liver-adipose MPS when cultured in PHM. Additionally, culturing the PNPLA3 mutant (M148) liver-adipose MPS in MHM induces IR and further increase steatosis, steatohepatitis, and fibrosis in the liver compared to PNPLA3 mutant systems cultured in PHM (71, 75). Similar steatosis, steatohepatitis, and fibrosis results occur in PNPLA3 mutant liver spheroids cultured in MHM and PHM. Clinical circulating biomarkers of NAFLD (AST, ALT, cytokeratin-18) are elevated in PNPLA3 mutant systems cultured in MHM compared to mutant systems in PHM. Moreover, proinflammatory and profibrotic crosstalk signaling molecule concentrations are elevated/dysregulated in PNPLA3 mutant systems cultured in MHM compared to mutant systems in PHM and those changes trend with clinical patient findings (e.g. IL-6 ↑, IL-1β ↑, leptin ↑, adiponectin ↓, fetuin A ↑, FGF21 ↑) (75, 76).

The PNPLA3 mutant MPS have differential transcriptome and metabolome profiles (PNPLA3 rs738409 vs. WT cultured in PHM and PNPLA3 in MHM vs. PNPLA3 in PHM), corresponding to the multi-hits in NAFLD progression. Hepatic lipid (TAG) accumulation and oxidative stress-induced pathway alterations are observed using intracellular metabolomics. Additionally dysregulated amino acid, lipid and carbohydrate metabolites occur in the mutant (M148) compared to WT (I148) (77).

Transcriptome analysis shows statistically significant differential expression in genes for de novo lipogenesis, inflammatory response, and ECM remodeling in disease phenotypes (i.e. cultured in MHM). By comparing the healthy and disease omics in each genotype, larger fold-change values occur for PNPLA3 mutant, indicating increased disease severity. Using NAFLD-Net, both common and differentially dysregulated pathways can be identified, and therapeutic targets for the two PNPLA3 genotypes (mutant and WT) can be identified. Small molecule and/or CRISPR-mediated perturbations of disease relevant pathways reduce the severity of the steatosis, fibrosis, and insulin resistance in the liver-adipose crosstalk MPSs. Disease severity reduction are less pronounced when compared to non-mutant MPSs cultured in MHM validating the importance of the stratified medicine MPS and setting the foundation for the identification of novel target pathways (i.e. stratified drug response) (78).

In an aspect, due to the high prevalence of the PNPLA3 rs738409 SNP in the NAFLD patient population (˜50% of patients), a suitable donor of hepatocytes and stellate cells for these experiments can be identified. However, as an alternative, PNPLA3 rs738409 adult hepatocytes, derived from induced pluripotent stem cells, are commercially available (DefiniGEN) and could be used for these experiments.

To maintain clinical relevance, the PNPLA3 mutant hepatocytes and stellate cells are patient-derived, and therefore, from a different genetic background compared to the WT (non-mutant) hepatocytes and stellate cells. While this aligns with the patient stratification approach and facilitates the identification of differentially regulated pathways, there are limitations on establishing causal relationships between the PNPLA3 mutation and disease status per se. To address this, the donor numbers evaluated are increased using this platform 115.

In an aspect, the role of pre- and post-menopause NAFL-NASH pathophysiology at the molecular and cellular levels is determined. The influence of biological sex, driven in large part by the sex hormones estradiol, progesterone, and testosterone (and their active metabolites), plays an important role in NAFL-NASH disease progression and severity. While the role of these hormones and their changing concentrations throughout the lifespan is characterized clinically, their role in novel drug target identification and patient stratification can be determined with the platforms 110, 115. The effects of estradiol and progesterone (female) on NAFLD severity and progression in the liver-adipose crosstalk MPS 120 are evaluated. This can establish female sex hormone-specific patient subpopulation stratified medicine MPSs for use in novel pathway identification and preclinical DDD.

Generally, pre- and post-menopausal concentrations of estradiol and progesterone (e.g., sex-hormone signaling) can drive differential effects on liver and adipose tissue physiology, and therefore, the NAFL-NASH disease profile in a mechanism-specific way. Further, while the NAFLD phenotype is similar in pre- and post-menopausal females, the underlying sex-based mechanistic disease drivers are sufficiently different as to identifiable by multi-omics analysis, and therefore, benefit from stratification and tailored therapeutic intervention strategies.

The prevalence and severity of NAFLD are greater in men (5×) and postmenopausal women (4×) than in premenopausal women suggesting a role for sex hormones in the disease etiology. Additionally, premature menopause and time since menopause directly increase the likelihood of increased fibrosis severity (adjusted cumulative odds ratio of 1.9 and 1.2, respectively). Specifically, estradiol reduces lipolysis and improves adipose tissue insulin sensitivity. Additional data indicates that estradiol inhibits stellate cell activation and reduces liver fibrosis. While the precise role (causal vs. correlative) of sex hormone binding globulin (SHBG) in NAFLD remains elucidated, there are studies indicating that circulating concentrations of SHBG are dysregulated in NAFLD patients. Finally, estradiol and progesterone differentially regulate genes pertaining to lipid metabolism including peroxisome proliferator-activated receptor alpha (PPARα), PPAR-γ, and farnesoid X receptor (FXR) and their targets in liver and adipose tissues (67, 85, 86). As compounds modulating these transcription factors' activity are currently being investigated as NAFLD therapeutics, it is important to examine the influence of biological sex on NAFLD progression and clinical patient stratification for drug treatment guidelines.

In this aspect, female sex hormones and SHBG are added to the MHM and PHM formulations to mimic the physiological concentrations present in premenopausal and postmenopausal females. The amount of estradiol and progesterone to add to the media are determined based on hormone binding coefficients to albumin and SHBG to achieve physiologically relevant concentrations of “free” hormone (e.g., available for binding to estrogen or progesterone receptors). To minimize the role of donor variability in these experiments, female single-donor cells are used for each cell type.

The platform 115 is used to characterize the phenotypic differences between pre- and post-menopausal females (hormone-based microenvironment) in NAFL-NASH disease progression using the liver-adipose MPS 120. Healthy premenopausal and postmenopausal female liver-adipose crosstalk MPSs are established as previously described. After an equilibration and maturation period (3 days), liver-adipose MPSs are switched either MHM or PHM and monitored for disease progression for up to 21 days. During the 21-day experiment, three periodic MPS takedowns (NAFL, NASH-fibrosis, NASH+fibrosis) are conducted to evaluate disease progression via phenotypic (histological and biomarker) analysis of the liver (steatosis and fibrosis) and adipose modules (steatosis). Additionally, RNA or intracellular metabolite extraction from the liver and adipose modules are conducted to determine the transcriptomic and metabolomic profiles associated with NAFL-NASH disease progression. At each takedown timepoint, cell culture medium are reserved for recirculating biomarker analysis including hepatokine, adipokine, and cytokine (crosstalk molecules) concentrations as previously described and shown in the preliminary data (e.g., graphs 300, 310, and 320 of FIGS. 3A-3C).

In this aspect, multi-omics data are generated using the liver-adipose MPS 120 and the NAFLD-Net 604 is implemented for female sex hormone-based stratified medicine and identification of therapeutic targets. Transcriptomics and intracellular metabolomics datasets are generated from pre- and post-menopausal MPSs for healthy and NAFLD phenotypes (e.g., cultured in MHM or PHM), and analyzed as previously described. Using the multi-tissue (liver-adipose) GEM framework NAFLD-Net 604, a context-specific multi-tissue network is construed as previously described in relation to FIG. 6. This framework is used for both pre- and post-menopausal phenotypes to assess the association of NAFLD progression and severity with female sex hormones. The therapeutic target lists are generated based on NAFLD-Net results and the targets prioritized as previously described. Prioritized targets are experimentally validated using CRISPR-mediated perturbation in spheroid cultures as described in relation to FIG. 4, and using the liver-adipose crosstalk MPS 120.

In an aspect, the disease modifying potential of pre- and post-menopausal subpopulation-specific targets is evaluated using small molecules and functional genomics (e.g., CRISPR activation or inhibition). Pre- and post-menopausal healthy (PHM) and NAFLD (MHM) liver and adipose spheroid cultures are established as previously described, and used to rapidly screen disease-modifying target pairs using small molecule and/or CRISPR sgRNA combinations. Specifically, the top-ranked disease modifying targets identified using the NAFLD-Net are investigated in combination with the most clinically advanced small molecules (e.g., elafibranor+CRISPR sgRNA targeting the top-ranked inflammatory signaling pathway) to evaluate the synergistic effects on disease phenotype.

The liver and adipose spheroids are treated with combinations of known small molecules (e.g., cenicriviroc, rosiglitazone, elafibranor, and OCA) that modulate the activity or expression of disease-relevant signaling pathways (e.g. CCR2-5, FXR, PPARγ, PPARβ/δ, and PPARγ) as previously described. In these screening experiments, the ability of the disease-modifying combinations to rescue the NAFLD phenotype are investigated using a dose-response approach.

The most efficacious small molecule and/or CRISPR treatment regimen (e.g., a disease-modifying target combination) are determined by evaluating the reduction in disease state (e.g., steatosis, fibrosis, inflammation, etc.) compared to untreated pre- and post-menopausal diseased spheroids.

These disease modifying targets are evaluated using the sex hormone-based crosstalk MPS to evaluate their disease modifying potentials. Accordingly, pre- and post-menopausal healthy and NAFLD liver-adipose MPSs are established as previously described. Following disease induction, the pre- and post-menopausal liver-adipose MPSs are treated with the most efficacious combinatorial treatment regimen (small molecule+CRISPR sgRNA) identified using the high-throughput screening spheroid model. The disease modifying potential of the therapeutic treatment regimens in the mutant liver-adipose crosstalk MPSs are determined using phenotypic (IR, steatosis, and fibrosis) and biomarker (apoptosis, hepatokine, adipokine, cytokine) readouts as previously described.

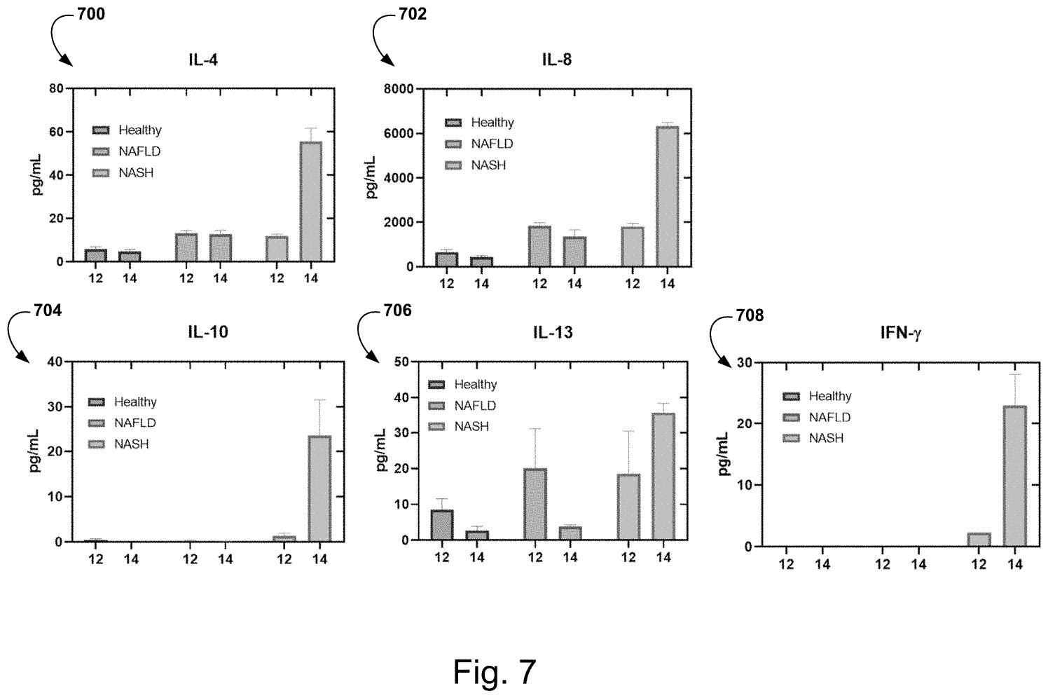

FIG. 7 shows examples of additional cytokine panel data. These are cytokines identified as important in NAFLD and/or NASH. These are generally involved in disease progression from NAFL to NASH. Graph 700 shows results for IL-4: profibrotic cytokine (drives TGF-beta expression). Graph 702 shows results for IL-8: proinflammatory and neutrophil recruitment. Graph 704 shows results for IL-10: profibrotic cytokine (drives TGF-beta expression). Graph 706 shows results for IL-13: profibrotic cytokine (drives TGF-beta expression). Graph 708 shows results for IFN-gamma: proinflammatory cytokine.

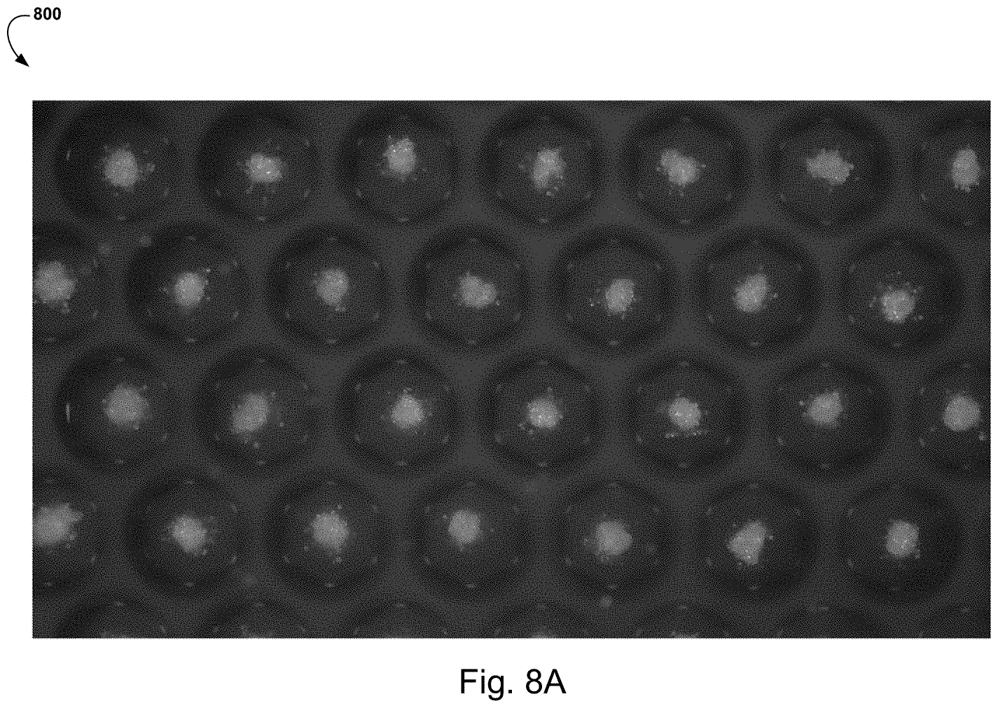

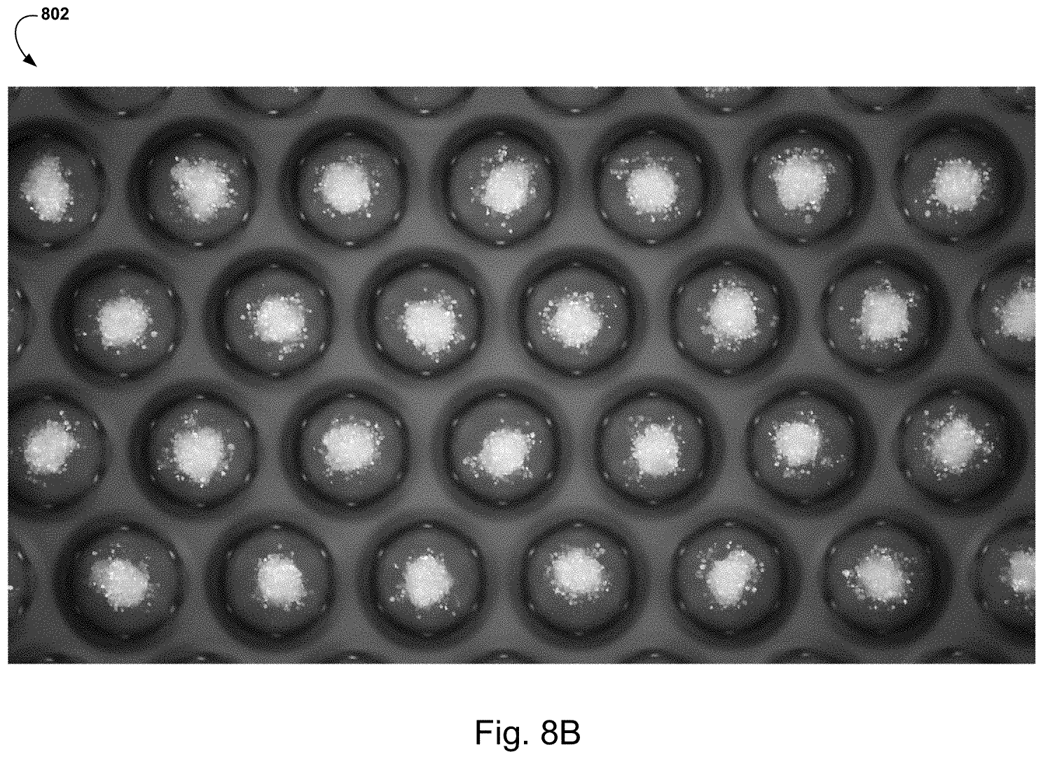

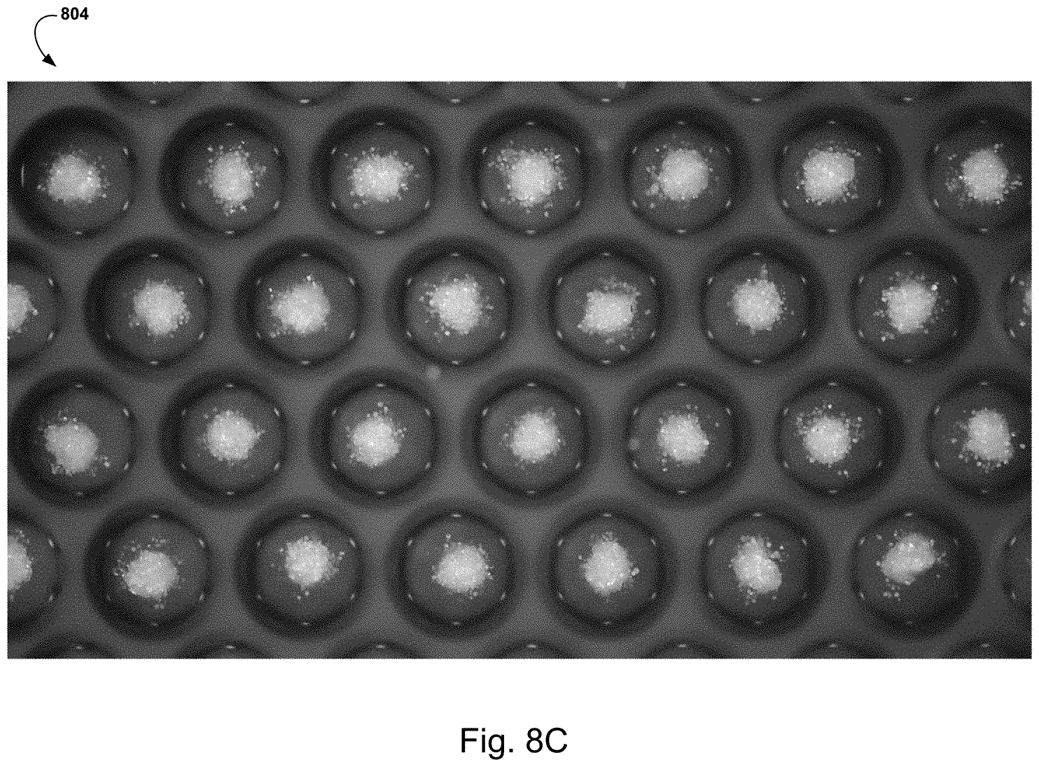

FIGS. 8A-8C show images 800, 802, and 804 representing green fluorescent protein signal intensity in untransfected liver spheroids compared to liver spheroids transfected with CRISPR-KO sgRNAs, and CRISPR-A sgRNAs, respectively. Image 800 shows the control image, image 802 shows the CRISPR-KO sgRNAs transfection, and image 804 shows CRISPR-A sgRNAs activation. These data indicate that liver cells can be transfected using functional genomic strategies (e.g., gene integration indicated by the GFP signal). In this example, CRISPR is used in two approaches: gene KO and gene activation. The SOP is translatable to RNA interference techniques using siRNAs or shRNAs. These data indicate the system can be perturbed using gain-of-function (GOF) and loss-of-function (LOF) screening strategies. GOF and LOF studies are important to investigating a gene's function in the context of disease and is usually only possible using germ-line edited animals or cells.

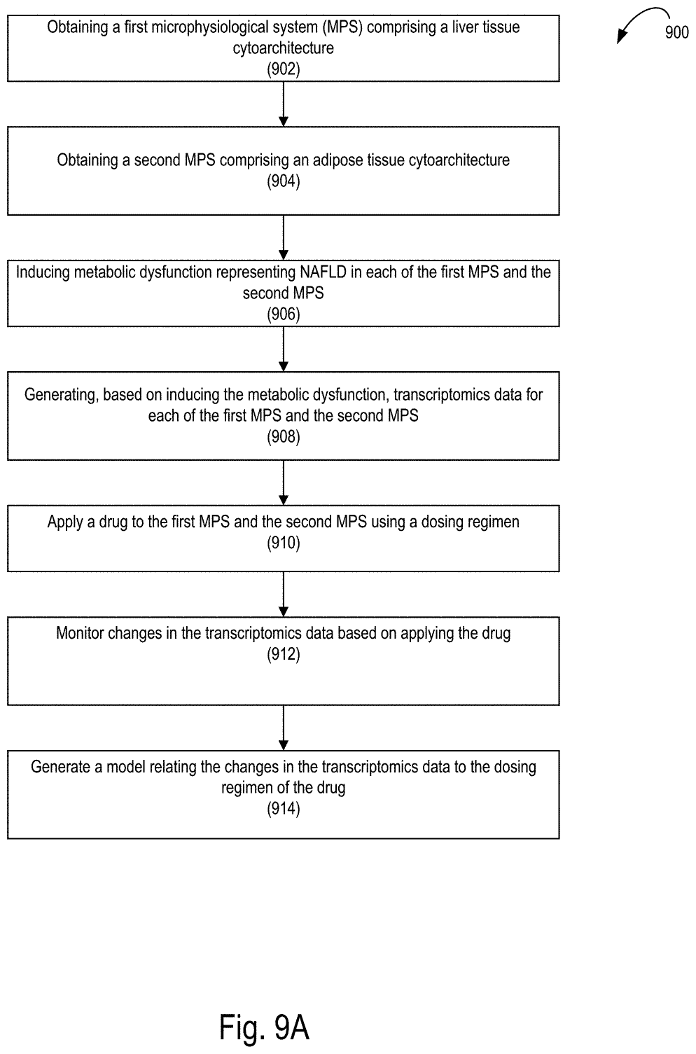

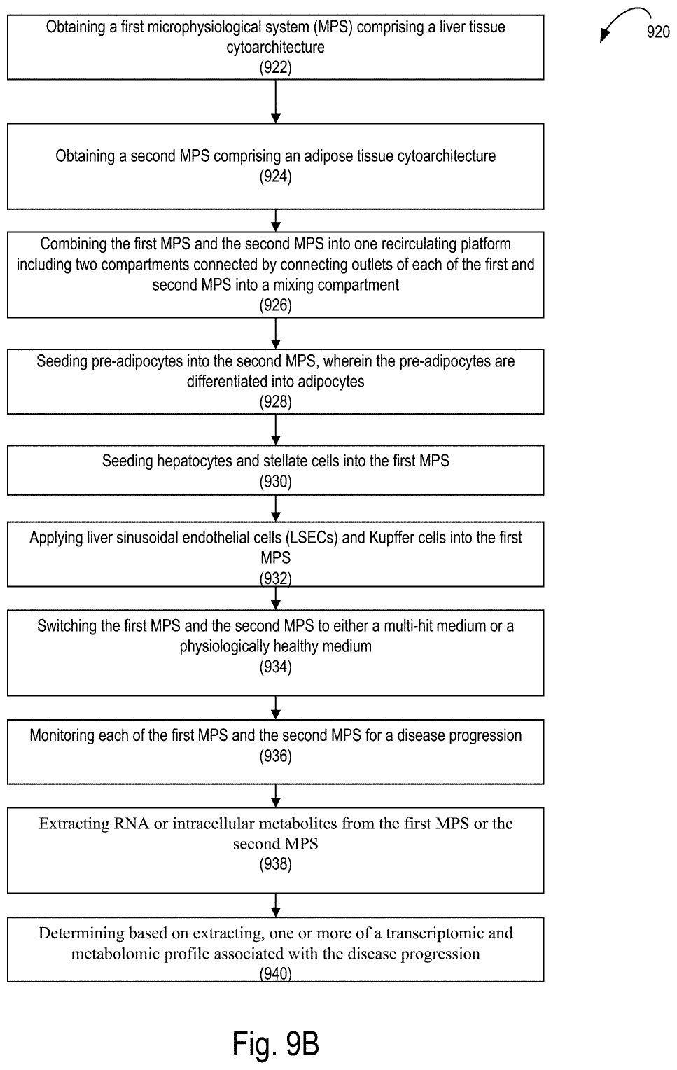

Turning to FIG. 9A, a process 900 for developing stratified medicine for nonalcoholic fatty liver disease (NAFLD) is shown. The process 900 includes obtaining (902) a first microphysiological system (MPS) comprising a liver tissue cytoarchitecture. The process 900 includes obtaining (904) a second MPS comprising an adipose tissue cytoarchitecture, the first MPS and the second MPS each comprising a compartment in a common fluidic system. Crosstalk occurs between the first MPS and the second MPS based on fluid flow in the common fluidic system. The process 900 includes inducing (906) metabolic dysfunction representing NAFLD in each of the first MPS and the second MPS. The process 900 includes generating (908), based on inducing the metabolic dysfunction, transcriptomics data for each of the first MPS and the second MPS. The process 900 includes applying (910) a drug to the first MPS and the second MPS using a dosing regimen. The process 900 includes monitoring (912) changes in the transcriptomics data based on applying the drug. The process 900 includes generating (914) a model relating the changes in the transcriptomics data to the dosing regimen of the drug.

In some implementations, the drug comprises one or more of rosiglitazone (PPARγ), elafibranor (PPARα and PPARβ/δ), obeticholic acid (OCA) (FXR), and cenicriviroc (CCR2-5). In some implementations, the drug comprises CRISPR short guide RNAs (sgRNAs) that modulate an activity or expression of disease-relevant signaling pathways. In some implementations, the sgRNAs comprise one or more of FXR, PPARγ, PPARβ/δ, PPARγ, and CCR2-5.

In some implementations, the dosing regimen comprises applying the drug at five concentrations spanning a nano-molar to milli-molar range. In some implementations, inducing metabolic dysfunction representing NAFLD in each of the first MPS and the second MPS comprises one or more of: inducing one or more of insulin resistance (IR), excessive de novo gluconeogenesis and lipogenesis, and dysregulated hepatokine signaling in the first MPS; and inducing one or more of IR, increased lipolysis, and dysregulated adipokine signaling in the second MPS.

In some implementations, inducing the metabolic dysfunction comprises: characterizing one or more of phenotypic, biomarker, and transcriptomic signatures of NAFLD pathology; and determining, a physiological relevance of one or more phenotypes, biomarkers, or transcriptomics of NAFLD.

In some implementations, applying the model to at least two stratified patient subpopulations based on differential biological mechanisms for each stratified patient subpopulation. In some implementations, the differential biological mechanism comprise one of a high-risk genetic single nucleotide polymorphism or a gender-specific hormone. In some implementations, the process 900 includes generating, based on applying the model, one or more of phenotypic, transcriptomic, and metabolomic datasets establishing a molecular characterization of each stratified patient subpopulation.

In some implementations, the process 900 includes connecting the first MPS and the second MPS by milli-fluidic recirculation to facilitate one or more of a hepatokine, an adipokine, and a cytokine crosstalk between the first MPS and the second MPS; and scaling each of the first MPS and the second MPS are each scaled based on a human physiology for one or more of the hepatokine, the adipokine, and the cytokine crosstalk. The human physiology represents comprises an oxygen-dependent liver metabolic zonation profile.

FIG. 9B shows a flow diagram including an example process 920 for developing stratified medicine for nonalcoholic fatty liver disease (NAFLD). The process 920 includes obtaining (922) a first microphysiological system (MPS) comprising a liver tissue cytoarchitecture. The process 920 includes obtaining (924) a second MPS comprising an adipose tissue cytoarchitecture. The process 920 includes combining (926) the first MPS and the second MPS into one recirculating platform including two compartments connected by connecting outlets of each of the first and second MPS into a mixing compartment. The process 920 includes seeding (928) pre-adipocytes into the second MPS, wherein the pre-adipocytes are differentiated into adipocytes. The process 920 includes seeding (930) hepatocytes and stellate cells into the first MPS. The process 920 includes applying (932) liver sinusoidal endothelial cells (LSECs) and Kupffer cells into the first MPS. The process 920 includes switching (934) the first MPS and the second MPS to either a multi-hit medium or a physiologically healthy medium. The process 920 includes monitoring (936) each of the first MPS and the second MPS for a disease progression. The process 920 includes extracting (938) RNA or intracellular metabolites from the first MPS or the second MPS. The process 920 includes determining (940), based on extracting, one or more of a transcriptomic and metabolomic profile associated with the disease progression.

In some implementations, the disease progression comprises nonalcoholic fatty liver (NAFL), nonalcoholic steatohepatitis (NASH)-fibrosis, or NASH+fibrosis.

In some implementations, the process 920 includes reconstructing one or more tissue-specific models for liver and adipose from global human metabolic network models. In some implementations, integrating one or more validated and functional liver-specific and adipose-specific genome-scale metabolic models (GEMs) through a media compartment connected to each of the first MPS and the second MPS. In some implementations, the process 920 includes generating a liver-adipose GEM representing a healthy metabolic state. In some implementations, the process 920 includes comparing the healthy metabolic state to a diseased state.

In some implementations, the diseased state comprises one or more of steatosis, steatohepatitis, and NASH with fibrosis phenotypes.

For MPS-to-human translation, to predict the in vivo gene expression from an in vitro (MPS) gene expression data using a regression model, at least one of the following algorithms can be used: lasso or elastic net regression, decision trees, and neural networks. ML algorithms are described in greater detail with respect to FIG. 11. The algorithm can translate disease modifications and target perturbations measured in the MPS to in vivo outcomes (effects). For example, the ML approach can be used to train and test a classifier based on at least one of the following algorithms: logistic regression, support vector machines, random forest, and neural networks. The classifier is trained using both datasets to classify healthy and disease phenotypes using transcriptomics data. Generally, the process 1100 includes evaluating and comparing performance of the models using the following metrics: area under receiver operating characteristic (ROC) curve (AUC) and root-mean-square-error (RMSE).

An example process for target discovery computational workflows can be as follows. In this example, systems biology (SB), quantitative systems pharmacology (QSP), or both can be used. Computational algorithms enable the system to perform mapping of high-content (-omics data) and high-throughput data from MPSs (e.g., MPS 110, 120 previously described) and characterization of molecular disease signatures. For target discovery efforts, a disease-Net computational framework is used. The computing system performs pathway-based integration of omics datasets with computational framework of genome-scale metabolic models (GEMs) to analyze disease pathophysiology.

The process includes constructing the GEMs. To do this, the computing system imports generic human metabolic network models from databases & public repositories (e.g., the gene expression omnibus (GEO)). The computing system refines and reconstructs the generic human metabolic network model for the cell/tissue types in the liver and adipose MPS by preserving the gene-protein-reaction associations using tissue-specificity information from databases (e.g., Human Protein Atlas, UniProt, etc.). The computing system validates the tissue-specific genome scale metabolic models (GEMs) by confirming network connectivity and running metabolic simulations under steady-state conditions. If there are at least two tissue- or cell-type-specific GEMs, the computing system integrates the GEMs by modeling a common pool compartment for nutrient of small molecule exchange, which mimics the culture media in any given in vitro experiment. The resulting integrated GEM is a scaffold on which to map MPS data.

The process includes generating MPS data. The computing system is configured to cause generation of transcriptomics data for gene expression using the MPS for healthy and (at least one disease stage of) disease phenotypes. Additionally, the computing system is configured to cause generation of intracellular metabolomics data using tissue samples cultured in the MPS.

The MPS data can be mapped on GEMs. Using the healthy transcriptomics data, the computing system validates the tissue-specific GEM by confirming network connectivity and running metabolic simulations under steady-state conditions. If (at least one stage of) disease is analyzed, the computing system maps the disease MPS data on the integrated GEM. The resulting validated integrated GEMs are referred to as a disease-Net framework. The computing system determines differential reactions in healthy and disease states, by comparing healthy and disease GEMs. The computing system determines the proteins (enzymes) that regulate these differential reactions and lists them as candidate therapeutic targets. If generated, the computing system integrates the MPS metabolomics data to validate the disease-Net framework by confirming that every experimentally measured metabolite is consumed and produced in the integrated GEMs. If generated, the computing system uses the MPS metabolomics data to validate the candidate therapeutic targets. The targets were determined based on transcriptomics data, and metabolomics facilitate confirmation as to whether an altered gene expression results in a perturbation in a reaction regulated by that gene's product (enzyme). The resulting list is called a “therapeutic target longlist”.

An example process for QSP-based computational target discovery is now described. The process includes training a health classifier. Training the classifier can include using both “healthy” and “disease” MPS data. Once trained, the classifier predicts whether a new set of phenotypic metrics were produced by a “healthy” or “disease” system. The process includes optimizing the QSP models. The QSP model includes mechanisms identified by omics analysis (e.g., healthy vs. disease MPS data from the MPSs 110, 120 previously described). The computing system optimizes parameters separately to fit phenotypic metrics from healthy MPS and the disease MPS. Once optimized, the QSP models produce the phenotypic metrics observed in the MPSs. The process includes predicting target effects. Each of the disease QSP model and the healthy QSP model are used to make predictions. The computing system is configured to simulate hypothetical target perturbations and identify those perturbations which transition a “disease” QSP model to a “healthy” class. The computing system proposes experiments to rescue healthy phenotypes for the “disease” MPS (e.g., an MPS 120 previously described).

At least some phenotypic metrics, but in some implementations, all of the phenotypic metrics, from both healthy and disease MPSs, are used to train a classifier (e.g., support vector machine, linear discriminant analysis, neural networks, etc.). The trained classifier will predict whether a new set of metrics represents a healthy or disease phenotype. Phenotypic data (described before), such as lipid accumulation, fibrosis, extracellular concentrations (cytokines, reactive oxygen species, cell death) are generated for healthy and disease MPSs.

The computing system constructs a mechanistic (ODE-based) QSP model to simulate experimental MPS data (phenotypic and -omics). A single set of equations (i.e., differential, algebraic, logic, and statistical) for the QSP model represents a set of causal physiological processes that may differ between the healthy and disease MPSs. Generally, two sets of parameters are obtained for the QSP model equations to capture the differences in the putative disease processes between healthy and disease MPSs. First, the QSP model parameters are adjusted using parameter estimation algorithms (e.g., non-linear least squares, Bayesian inference, evolutionary algorithms, etc.) to make the simulated phenotypic metrics match the phenotypic metrics from the healthy MPS. Those parameters, along with the QSP equations, define the healthy QSP model. Independently, the QSP model parameters are adjusted to fit the phenotypic metrics from the disease MPS. Those parameters, along with the same equations, define the disease QSP model.

The computing system is configured to simulate computational target perturbations in the QSP model to evaluate targets that can improve the disease phenotype. The same perturbations are applied to the healthy and disease QSP models, and the phenotypic output is classified using the same classifier that is trained on the MPS data. The computing system identifies perturbations that transition the disease QSP model from disease to healthy class without transitioning the class of the healthy QSP model. Simulated perturbations may represent target manipulations directly, like chemical (e.g., receptor agonists, antagonists) or physical (electrical and optical) perturbations. Successful simulated perturbations directly predict target effects that can rescue the healthy phenotype in the disease MPS. Alternatively, simulated perturbations can represent more abstract phenomenological changes (e.g., changes to biophysical properties, like membrane capacitance). More detailed QSP models are constructed subsequently to simulate mechanistic target manipulations that cause the same effects identified using the more abstract phenomenological model. Experiments predicted by QSP simulation to improve phenotype in the disease MPS can be tested in the high-throughput workflow.

A computing system applies the QSP models for target prioritization. For example, if the “therapeutic target longlist” is too long or not ordered, targets can be rank-ordered based on several criteria, such as tissue-specificity, druggability, efficacy/safety, novelty, etc. A scoring system uses the abovementioned criteria to rank-order the therapeutic targets to be tested experimentally. Databases (e.g., DrugBank, Therapeutic Target Database (TTD), Open Targets, PHAROS, etc.) are referenced to extract druggability, and efficacy/safety information for each target. Based on the information from the databases and the target effects predicted by QSP simulation (e.g., health class transitions), the computing system assigns to undruggable and toxicity-inducing targets negative scores (e.g., lower scores) for these criteria, while druggable, safe and disease modifying targets are assigned positive scores (e.g., higher scores). If information is missing for a criterion, no score is assigned. The therapeutic target longlist can be rank-ordered based on the scores and target validation experiments are proposed based on this prioritized target list (target shortlist).

For experimental target validation, shortlisted therapeutic targets (either mono or combo targets) are perturbed in healthy and disease MPS to validate (evaluate) their disease modifying potential. The target perturbations may include xenobiotics, biologics, RNAi, CRISPR sgRNA addition to healthy or disease liver and adipose MPS. Generally, phenotypic metrics are quantified to assess disease modification. In some instances, multi-omics datasets can be generated and quantified to assess or confirm disease modification.

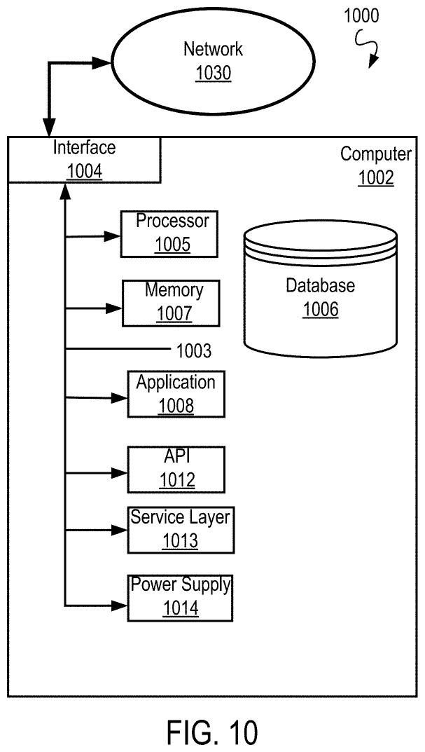

FIG. 10 is a block diagram of an example computer system 1000 used to provide computational functionalities associated with described algorithms, methods, functions, processes, flows, and procedures described in the present disclosure (such as the method 200 described previously with reference to FIG. 2), according to some implementations of the present disclosure. The illustrated computer 1002 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 1002 can include input devices such as keypads, keyboards, and touch screens that can accept user information. Also, the computer 1002 can include output devices that can convey information associated with the operation of the computer 1002. The information can include digital data, visual data, audio information, or a combination of information. The information can be presented in a graphical user interface (UI or GUI).

The computer 1002 can serve in a role as a client, a network component, a server, a database, a persistency, or components of a computer system for performing the subject matter described in the present disclosure. The illustrated computer 1002 is communicably coupled with a network 1030. In some implementations, one or more components of the computer 1002 can be configured to operate within different environments, including cloud-computing-based environments, local environments, global environments, and combinations of environments.

At a high level, the computer 1002 is an electronic computing device operable to receive, transmit, process, store, and manage data and information associated with the described subject matter. According to some implementations, the computer 1002 can also include, or be communicably coupled with, an application server, an email server, a web server, a caching server, a streaming data server, or a combination of servers.

The computer 1002 can receive requests over network 1030 from a client application (for example, executing on another computer 1002). The computer 1002 can respond to the received requests by processing the received requests using software applications. Requests can also be sent to the computer 1002 from internal users (for example, from a command console), external (or third) parties, automated applications, entities, individuals, systems, and computers.

Each of the components of the computer 1002 can communicate using a system bus 1003. In some implementations, any or all of the components of the computer 1002, including hardware or software components, can interface with each other or the interface 1004 (or a combination of both), over the system bus 1003. Interfaces can use an application programming interface (API) 1012, a service layer 1013, or a combination of the API 1012 and service layer 1013. The API 1012 can include specifications for routines, data structures, and object classes. The API 1012 can be either computer-language independent or dependent. The API 1012 can refer to a complete interface, a single function, or a set of APIs.

The service layer 1013 can provide software services to the computer 1002 and other components (whether illustrated or not) that are communicably coupled to the computer 1002. The functionality of the computer 1002 can be accessible for all service consumers using this service layer. Software services, such as those provided by the service layer 1013, can provide reusable, defined functionalities through a defined interface. For example, the interface can be software written in JAVA, C++, or a language providing data in extensible markup language (XML) format. While illustrated as an integrated component of the computer 1002, in alternative implementations, the API 1012 or the service layer 1013 can be stand-alone components in relation to other components of the computer 1002 and other components communicably coupled to the computer 1002. Moreover, any or all parts of the API 1012 or the service layer 1013 can be implemented as child or sub-modules of another software module, enterprise application, or hardware module without departing from the scope of the present disclosure.

The computer 1002 includes an interface 1004. Although illustrated as a single interface 1004 in FIG. 10, two or more interfaces 1004 can be used according to particular needs, desires, or particular implementations of the computer 1002 and the described functionality. The interface 1004 can be used by the computer 1002 for communicating with other systems that are connected to the network 1030 (whether illustrated or not) in a distributed environment. Generally, the interface 1004 can include, or be implemented using, logic encoded in software or hardware (or a combination of software and hardware) operable to communicate with the network 1030. More specifically, the interface 1004 can include software supporting one or more communication protocols associated with communications. As such, the network 1030 or the interface's hardware can be operable to communicate physical signals within and outside of the illustrated computer 1002.

The computer 1002 includes a processor 1005. Although illustrated as a single processor 1005 in FIG. 10, two or more processors 1005 can be used according to particular needs, desires, or particular implementations of the computer 1002 and the described functionality. Generally, the processor 1005 can execute instructions and can manipulate data to perform the operations of the computer 1002, including operations using algorithms, methods, functions, processes, flows, and procedures as described in the present disclosure.

The computer 1002 also includes a database 1006 that can hold data for the computer 1002 and other components connected to the network 1030 (whether illustrated or not). For example, database 1006 can be an in-memory, conventional, or a database storing data consistent with the present disclosure. In some implementations, database 1006 can be a combination of two or more different database types (for example, hybrid in-memory and conventional databases) according to particular needs, desires, or particular implementations of the computer 1002 and the described functionality. Although illustrated as a single database 1006 in FIG. 10, two or more databases (of the same, different, or combination of types) can be used according to particular needs, desires, or particular implementations of the computer 1002 and the described functionality. While database 1006 is illustrated as an internal component of the computer 1002, in alternative implementations, database 1006 can be external to the computer 1002.

The computer 1002 also includes a memory 1007 that can hold data for the computer 1002 or a combination of components connected to the network 1030 (whether illustrated or not). Memory 1007 can store any data consistent with the present disclosure. In some implementations, memory 1007 can be a combination of two or more different types of memory (for example, a combination of semiconductor and magnetic storage) according to particular needs, desires, or particular implementations of the computer 1002 and the described functionality. Although illustrated as a single memory 1007 in FIG. 10, two or more memories 1007 (of the same, different, or combination of types) can be used according to particular needs, desires, or particular implementations of the computer 1002 and the described functionality. While memory 1007 is illustrated as an internal component of the computer 1002, in alternative implementations, memory 1007 can be external to the computer 1002.

The application 1008 can be an algorithmic software engine providing functionality according to particular needs, desires, or particular implementations of the computer 1002 and the described functionality. For example, application 1008 can serve as one or more components, modules, or applications. Further, although illustrated as a single application 1008, the application 1008 can be implemented as multiple applications 1008 on the computer 1002. In addition, although illustrated as internal to the computer 1002, in alternative implementations, the application 1008 can be external to the computer 1002.

The computer 1002 can also include a power supply 1014. The power supply 1014 can include a rechargeable or non-rechargeable battery that can be configured to be either user- or non-user-replaceable. In some implementations, the power supply 1014 can include power-conversion and management circuits, including recharging, standby, and power management functionalities. In some implementations, the power-supply 1014 can include a power plug to allow the computer 1002 to be plugged into a wall socket or a power source to, for example, power the computer 1002 or recharge a rechargeable battery.

There can be any number of computers 1002 associated with, or external to, a computer system containing computer 1002, with each computer 1002 communicating over network 1030. Further, the terms “client,” “user,” and other appropriate terminology can be used interchangeably, as appropriate, without departing from the scope of the present disclosure. Moreover, the present disclosure contemplates that many users can use one computer 1002 and one user can use multiple computers 1002.

FIG. 11 is a diagram illustrating an example computer system 1100 configured to execute a machine learning model. Generally, the computer system 1100 is configured to process data indicating a phenotype and determine a class label (“healthy” or “disease”) to predict the effect of a target on the state of the MPS. The system 1100 includes computer processors 1110. The computer processors 1110 include computer-readable memory 1111 and computer readable instructions 1112. The system 1100 also includes a machine learning system 1150. The machine learning system 1150 includes a machine learning model 1120. The machine learning model 1120 can be separate from or integrated with the computer processors 1110.

The computer-readable medium 1111 (or computer-readable memory) can include any data storage technology type which is suitable to the local technical environment, including but not limited to semiconductor based memory devices, magnetic memory devices and systems, optical memory devices and systems, fixed memory, removable memory, disc memory, flash memory, dynamic random-access memory (DRAM), static random-access memory (SRAM), electronically erasable programmable read-only memory (EEPROM) and the like. In an embodiment, the computer-readable medium 1111 includes code-segment having executable instructions.

In some implementations, the computer processors 1110 include a general purpose processor. In some implementations, the computer processors 1110 include a central processing unit (CPU). In some implementations, the computer processors 1110 include at least one application specific integrated circuit (ASIC). The computer processors 1110 can also include general purpose programmable microprocessors, graphic processing units, special-purpose programmable microprocessors, digital signal processors (DSPs), programmable logic arrays (PLAs), field programmable gate arrays (FPGA), special purpose electronic circuits, etc., or a combination thereof. The computer processors 1110 are configured to execute program code means such as the computer-executable instructions 1112 and configured to execute executable logic that includes the machine learning model 1120.

The computer processors 1110 are configured to receive data indicating a molecular structure of, for example, a drug. The data can be obtained through one or more means, such as wireless communications with databases, optical fiber communications, USB, CD-ROM, and so forth.

The machine learning model 1120 is capable of processing the data to determine the class of phenotype (“healthy or disease”). In some implementations, the machine learning model 1120 is trained to determine class using a data set that includes MPS data (e.g., phenotypic, transcriptomic, etc.) and MPS labels. The machine learning model 1120 can classify the phenotype predicted by in vitro or in silico target perturbations. Accordingly, when a data set (in vitro or in silico) is introduced to the machine learning model 1120, it can predict whether an MPS platform exhibits a healthy or disease phenotype.

The machine learning system 1150 is capable of applying machine learning techniques to train the machine learning model 1120. As part of the training of the machine learning model 1120, the machine learning system 1150 forms a training set of input data by identifying a positive training set of input data items that have been determined to have the property in question, and, in some embodiments, forms a negative training set of input data items that lack the property in question.

The machine learning system 1150 extracts feature values from the input data of the training set, the features being variables deemed potentially relevant to whether or not the input data items have the associated property or properties. An ordered list of the features for the input data is herein referred to as the feature vector for the input data. In one embodiment, the machine learning system 1150 applies dimensionality reduction (e.g., via linear discriminant analysis (LDA), principle component analysis (PCA), or the like) to reduce the amount of data in the feature vectors for the input data to a smaller, more representative set of data.

In some implementations, the machine learning system 1150 uses supervised machine learning to train the machine learning models 1120 with the feature vectors of the positive training set and the negative training set serving as the inputs. Different machine learning techniques—such as linear support vector machine (linear SVM), boosting for other algorithms (e.g., AdaBoost), neural networks, logistic regression, naïve Bayes, memory-based learning, random forests, bagged trees, decision trees, boosted trees, or boosted stumps—may be used in different embodiments. The machine learning model 1120, when applied to the feature vector extracted from the input data item, outputs an indication of whether the input data item has the property in question, such as a Boolean yes/no estimate, or a scalar value representing a probability.

In some embodiments, a validation set is formed of additional input data, other than those in the training sets, which have already been determined to have or to lack the property in question. The machine learning system 1150 applies the trained machine learning model 1120 to the data of the validation set to quantify the accuracy of the machine learning model 1120. Common metrics applied in accuracy measurement include: Precision=TP/(TP+FP) and Recall=TP/(TP+FN), where precision is how many the machine learning model correctly predicted (TP or true positives) out of the total it predicted (TP+FP or false positives), and recall is how many the machine learning model correctly predicted (TP) out of the total number of input data items that did have the property in question (TP+FN or false negatives). The F score (F-score=2*PR/(P+R)) unifies precision and recall into a single measure. In one embodiment, the machine learning module iteratively re-trains the machine learning model until the occurrence of a stopping condition, such as the accuracy measurement indication that the model is sufficiently accurate, or a number of training rounds having taken place.

In some implementations, the machine learning model 1120 is a convolutional neural network (CNN). A CNN can be configured based on a presumption that inputs to the CNN correspond to image pixel data for an image or other data that includes features at multiple spatial locations. For example, sets of inputs can form a multi-dimensional data structure, such as a tensor, that represent color features of an example digital image (e.g., a biological image of biological tissue). In some implementations, inputs to the CNN correspond to a variety of other types of data, such as data obtained from different devices and sensors of a vehicle, point cloud data, audio data that includes certain features or raw audio at each of multiple time steps, or various types of one-dimensional or multiple dimensional data. A convolutional layer of the CNN can process the inputs to transform features of the image that are represented by inputs of the data structure. For example, the inputs are processed by performing dot product operations using input data along a given dimension of the data structure and a set of parameters for the convolutional layer.

Performing computations for a convolutional layer can include applying one or more sets of kernels to portions of inputs in the data structure. The manner in which CNN performs the computations can be based on specific properties for each layer of an example multi-layer neural network or deep neural network that supports deep neural net workloads. A deep neural network can include one or more convolutional towers (or layers) along with other computational layers. In particular, for example computer vision applications, these convolutional towers often account for a large proportion of the inference calculations that are performed. Convolutional layers of a CNN can have sets of artificial neurons that are arranged in three dimensions, a width dimension, a height dimension, and a depth dimension. The depth dimension corresponds to a third dimension of an input or activation volume and can represent respective color channels of an image. For example, input images can form an input volume of data (e.g., activations), and the volume has dimensions 32×32×3 (width, height, depth respectively). A depth dimension of 3 can correspond to the RGB color channels of red (R), green (G), and blue (B).

In general, layers of a CNN are configured to transform the three dimensional input volume (inputs) to a multi-dimensional output volume of neuron activations (activations). For example, a 3D input structure of 32×32×3 holds the raw pixel values of an example image, in this case an image of width 32, height 32, and with three color channels, R, G, B. A convolutional layer of a CNN of the machine learning model 1120 computes the output of neurons that may be connected to local regions in the input volume. Each neuron in the convolutional layer can be connected only to a local region in the input volume spatially, but to the full depth (e.g., all color channels) of the input volume. For a set of neurons at the convolutional layer, the layer computes a dot product between the parameters (weights) for the neurons and a certain region in the input volume to which the neurons are connected. This computation may result in a volume such as 32×32×12, where 12 corresponds to a number of kernels that are used for the computation. A neuron's connection to inputs of a region can have a spatial extent along the depth axis that is equal to the depth of the input volume. The spatial extent corresponds to spatial dimensions (e.g., x and y dimensions) of a kernel.

A set of kernels can have spatial characteristics that include a width and a height and that extends through a depth of the input volume. Each set of kernels for the layer is applied to one or more sets of inputs provided to the layer. That is, for each kernel or set of kernels, the machine learning model 1120 can overlay the kernel, which can be represented multi-dimensionally, over a first portion of layer inputs (e.g., that form an input volume or input tensor), which can be represented multi-dimensionally. For example, a set of kernels for a first layer of a CNN may have size 5×5×3×16, corresponding to a width of 5 pixels, a height of 5 pixel, a depth of 3 that corresponds to the color channels of the input volume to which to a kernel is being applied, and an output dimension of 16 that corresponds to a number of output channels. In this context, the set of kernels includes 16 kernels so that an output of the convolution has a depth dimension of 16.

The machine learning model 1120 can then compute a dot product from the overlapped elements. For example, the machine learning model 1120 can convolve (or slide) each kernel across the width and height of the input volume and compute dot products between the entries of the kernel and inputs for a position or region of the image. Each output value in a convolution output is the result of a dot product between a kernel and some set of inputs from an example input tensor. The dot product can result in a convolution output that corresponds to a single layer input, e.g., an activation element that has an upper-left position in the overlapped multi-dimensional space. As discussed above, a neuron of a convolutional layer can be connected to a region of the input volume that includes multiple inputs. The machine learning model 1120 can convolve each kernel over each input of an input volume. The machine learning model 1120 can perform this convolution operation by, for example, moving (or sliding) each kernel over each input in the region.

The machine learning model 1120 can move each kernel over inputs of the region based on a stride value for a given convolutional layer. For example, when the stride is set to 1, then the machine learning model 1120 can move the kernels over the region one pixel (or input) at a time. Likewise, when the stride is 2, then the machine learning model 1120 can move the kernels over the region two pixels at a time. Thus, kernels may be shifted based on a stride value for a layer and the machine learning model 1120 can repeatedly perform this process until inputs for the region have a corresponding dot product. Related to the stride value is a skip value. The skip value can identify one or more sets of inputs (2×2), in a region of the input volume, that are skipped when inputs are loaded for processing at a neural network layer. In some implementations, an input volume of pixels for an image can be “padded” with zeros, e.g., around a border region of an image. This zero-padding is used to control the spatial size of the output volumes.

As discussed previously, a convolutional layer of CNN is configured to transform a three dimensional input volume (inputs of the region) to a multi-dimensional output volume of neuron activations. For example, as the kernel is convolved over the width and height of the input volume, the machine learning model 1120 can produce a multi-dimensional activation map that includes results of convolving the kernel at one or more spatial positions based on the stride value. In some cases, increasing the stride value produces smaller output volumes of activations spatially. In some implementations, an activation can be applied to outputs of the convolution before the outputs are sent to a subsequent layer of the CNN.

An example convolutional layer can have one or more control parameters for the layer that represent properties of the layer. For example, the control parameters can include a number of kernels, K, the spatial extent of the kernels, F, the stride (or skip), S, and the amount of zero padding, P. Numerical values for these parameters, the inputs to the layer, and the parameter values of the kernel for the layer shape the computations that occur at the layer and the size of the output volume for the layer. In some implementations, the spatial size of the output volume is computed as a function of the input volume size, W, using the formula (W−F+2P)/S+1. For example, an input tensor can represent a pixel input volume of size [227×227×3]. A convolutional layer of a CNN can have a spatial extent value of F=11, a stride value of S=4, and no zero-padding (P=0). Using the above formula and a layer kernel quantity of K=116, the machine learning model 1120 performs computations for the layer that results in a convolutional layer output volume of size [55×55×156], where 55 is obtained from [(227−11+0)/4+1=55].

The computations (e.g., dot product computations) for a convolutional layer, or other layers, of a CNN involve performing mathematical operations, e.g., multiplication and addition, using a computation unit of a hardware circuit of the machine learning model 1120. The design of a hardware circuit can cause a system to be limited in its ability to fully utilize computing cells of the circuit when performing computations for layers of a neural network.

In the previous description, for the purposes of explanation, numerous specific details are set forth in order to provide a thorough understanding of the present disclosure. However, the present disclosure may be practiced without these specific details. In other instances, well-known structures and devices are shown in block diagram form in order to avoid unnecessarily obscuring the present disclosure.

In the drawings, specific arrangements or orderings of schematic elements, such as those representing devices, modules, instruction blocks and data elements, are shown for ease of description. However, it should be understood by those skilled in the art that the specific ordering or arrangement of the schematic elements in the drawings is not meant to imply that a particular order or sequence of processing, or separation of processes, is required. Further, the inclusion of a schematic element in a drawing is not meant to imply that such element is required in all embodiments or that the features represented by such element may not be included in or combined with other elements in some implementations.

Further, in the drawings, where connecting elements, such as solid or dashed lines or arrows, are used to illustrate a connection, relationship, or association between or among two or more other schematic elements, the absence of any such connecting elements is not meant to imply that no connection, relationship, or association can exist. In other words, some connections, relationships, or associations between elements are not shown in the drawings so as not to obscure the disclosure. In addition, for ease of illustration, a single connecting element is used to represent multiple connections, relationships or associations between elements. For example, where a connecting element represents a communication of signals, data, or instructions, it should be understood by those skilled in the art that such element represents one or multiple signal paths (e.g., a bus), as may be needed, to affect the communication.

Reference is made in detail to implementations, examples of which are illustrated in the accompanying drawings. In the previous description, numerous specific details are set forth in order to provide a thorough understanding of the various described implementations. However, it are apparent to one of ordinary skill in the art that the various described implementations may be practiced without these specific details. In other instances, well-known methods, procedures, components, circuits, and networks have not been described in detail so as not to unnecessarily obscure aspects of the implementations.

Several features are described that can each be used independently of one another or with any combination of other features. However, any individual feature may not address any of the problems discussed above or might only address one of the problems discussed above. Some of the problems discussed above might not be fully addressed by any of the features described in this specification. Although headings are provided, data related to a particular heading, but not found in the section having that heading, may also be found elsewhere in this description.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back end component (for example, as a data server), or that includes a middleware component (for example, an application server). Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

In the foregoing description, embodiments of the invention have been described with reference to numerous specific details that may vary from implementation to implementation. The description and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising” or “further including” in the foregoing description or following claims, what follows this phrase can be an additional step or entity, or a sub-step/sub-entity of a previously-recited step or entity.

Particular implementations of the subject matter have been described. Other implementations, alterations, and permutations of the described implementations are within the scope of the following claims as are apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of embodiments of these systems and methods have been described. Nevertheless, it are understood that various modifications may be made without departing from the spirit and scope of this disclosure.

What is claimed is:

1. A method for developing stratified medicine for nonalcoholic fatty liver disease (NAFLD), the method comprising:
obtaining a first microphysiological system (MPS) comprising a liver tissue cytoarchitecture;
obtaining a second MPS comprising an adipose tissue cytoarchitecture, the first MPS and the second MPS each comprising a compartment in a common fluidic system, wherein crosstalk occurs between the first MPS and the second MPS based on fluid flow in the common fluidic system;
inducing metabolic dysfunction representing NAFLD in each of the first MPS and the second MPS;
generating, based on inducing the metabolic dysfunction, transcriptomics data for each of the first MPS and the second MPS;
applying a drug to the first MPS and the second MPS using a dosing regimen;
monitoring changes in the transcriptomics data based on applying the drug; and
generating a model relating the changes in the transcriptomics data to the dosing regimen of the drug.

2. The method of claim 1, wherein the drug comprises one or more of small and large molecules configured to modulate activity of disease-relevant signaling pathways.

3. The method of claim 2, wherein the one or more of small and large molecules comprise one or more of rosiglitazone (PPARγ), elafibranor (PPARα and PPARβ/δ), obeticholic acid (OCA) (FXR), and cenicriviroc (CCR2-5).

4. The method of claim 1, wherein the drug comprises CRISPR short guide RNAs (sgRNAs) that modulate an activity or expression of disease-relevant signaling pathways.

5. The method of claim 4, wherein the sgRNAs comprise one or more of FXR, PPARα, PPARβ/δ, PPARγ, and CCR2-5.

6. The method of claim 1, wherein the dosing regimen comprises applying the drug at five concentrations spanning a nano-molar to milli-molar range.

7. The method of claim 1, wherein inducing metabolic dysfunction representing NAFLD in each of the first MPS and the second MPS comprises one or more of:
inducing one or more of insulin resistance (IR), excessive de novo gluconeogenesis and lipogenesis, and dysregulated hepatokine signaling in the first MPS; and
inducing one or more of IR, increased lipolysis, and dysregulated adipokine signaling in the second MPS.

8. The method of claim 1, wherein inducing the metabolic dysfunction comprises:
characterizing one or more of phenotypic, biomarker, and transcriptomic signatures of NAFLD pathology; and
determining, a physiological relevance of one or more phenotypes, biomarkers, or transcriptomics of NAFLD.

9. The method of claim 1, further comprising:
applying the model to one or more stratified patient subpopulations based on differential biological mechanisms for each stratified patient subpopulation.

10. The method of claim 9, wherein the differential biological mechanism comprises one of a high-risk genetic single nucleotide polymorphism or a gender-specific hormone.

11. The method of claim 9, further comprising:
generating, based on applying the model, one or more of phenotypic, transcriptomic, and metabolomic datasets establishing a molecular characterization of each stratified patient subpopulation.

12. The method of claim 1, further comprising:
connecting the first MPS and the second MPS by milli-fluidic recirculation to facilitate one or more of a hepatokine, an adipokine, and a cytokine crosstalk between the first MPS and the second MPS; and
scaling each of the first MPS and the second MPS are each scaled based on a human physiology for one or more of the hepatokine, the adipokine, and the cytokine crosstalk.

13. The method of claim 12, wherein the human physiology represents comprises an oxygen-dependent liver metabolic zonation profile.

14. A method for developing stratified medicine for nonalcoholic fatty liver disease (NAFLD), the method comprising:
obtaining a first microphysiological system (MPS) comprising a liver tissue cytoarchitecture;
obtaining a second MPS comprising an adipose tissue cytoarchitecture;
combining the first MPS and the second MPS into one recirculating platform including two compartments connected by connecting outlets of each of the first and second MPS into a mixing compartment;
seeding pre-adipocytes into the second MPS, wherein the pre-adipocytes are differentiated into adipocytes;
seeding hepatocytes and stellate cells into the first MPS;
applying liver sinusoidal endothelial cells (LSECs) and Kupffer cells into the first MPS;
switching the first MPS and the second MPS to either a medium including disease-inducing factors or to a physiologically healthy medium;
monitoring each of the first MPS and the second MPS for a disease progression; and
extracting RNA or intracellular metabolites from the first MPS or the second MPS; and
determining, based on extracting, one or more of a transcriptomic and metabolomic profile associated with the disease progression.

15. The method of claim 14, wherein the disease progression comprises nonalcoholic fatty liver (NAFL), nonalcoholic steatohepatitis (NASH)-fibrosis, or NASH and fibrosis.

16. The method of claim 14, further comprising:
reconstructing one or more tissue-specific models for liver and adipose from global human metabolic network models.

17. The method of claim 14, further comprising:
integrating one or more validated and functional liver-specific and adipose-specific genome-scale metabolic models (GEMs) through a media compartment connected to each of the first MPS and the second MPS;
generating a liver-adipose GEM representing a healthy metabolic state; and
comparing the healthy metabolic state to a diseased state.

18. The method of claim 17, wherein the diseased state comprises one or more of steatosis, steatohepatitis, and NASH with fibrosis phenotypes.

19. The method of claim 16, further comprising:
identifying a target from the GEM;
perturbing the target with small molecules; and
evaluating a change in a phenotype of the target to determine whether NAFLD is improved.

20. A method for developing stratified medicine for nonalcoholic fatty liver disease (NAFLD), the method comprising:
obtaining a microphysiological system (VIPS) comprising a liver tissue cytoarchitecture;
inducing metabolic dysfunction representing NAFLD in the liver tissue of the MPS;
generating, based on inducing the metabolic dysfunction, transcriptomics data for the MPS;
applying a drug to the MPS using a dosing regimen;
monitoring changes in the transcriptomics data based on applying the drug; and
generating a model relating the changes in the transcriptomics data to the dosing regimen of the drug.
